(19) United States
(12) Patent Application Publication
(10) Pub. No.: US 2024/0000789 A1
(43) Pub. Date: Jan. 4, 2024

(54) THERAPEUTIC COMBINATIONS COMPRISING A CRAF INHIBITOR

(71) Applicant: NOVARTIS AG, Basel (CH)

(72) Inventors: Giordano Caponigro, Foxboro, MA (US); Vesselina Cooke, Newton, MA (US); Uz Martin Stammberger, Weiler b. Bingen (DE); Darrin Stuart, Marblehead, MA (US)

(21) Appl. No.: 17/998,391

(22) PCT Filed: May 11, 2021
(86) PCT No.: PCT/IB2021/054013

§ 371 Date: Nov. 10, 2022

Related U.S. Application Data
(60) Provisional application No. 63/023,470, filed on May 12, 2020.

(51) Int. Cl. A61K 31/5377; A61K 31/4965; A61K 31/519; A61P 35/00; A61P 35/04

(52) U.S. Cl. CPC A61K 31/5377; A61K 31/4965; A61P 35/04; A61P 35/00; A61K 31/519

(57) ABSTRACT

The present invention provides a pharmaceutical combination comprising a CRAF inhibitor in combination with (i) an ERK inhibitor or (ii) a MEK inhibitor or (iii) a CDK4/6 inhibitor, each as defined herein, or independently in each case a pharmaceutically acceptable salt thereof, for use in the treatment of NRAS-mutant melanoma and in the treatment of BRAF-mutant melanoma. wherein the melanoma may be unresectable and/or metastatic melanoma.

### RELATED APPLICATIONS

This application is a § 371 U.S. National Stage Entry of International Application No. PCT/IB2021/054013, filed May 11, 2021, which claims the benefit of U.S. Provisional Application Serial No. 63/023,470, filed May 12, 2020, each of which are hereby incorporated by reference in their entirety.

### TECHNICAL FIELD

The present invention provides a pharmaceutical combination comprising naporafenib (the Compound of formula (I) or Compound A), as defined herein, or a pharmaceutically acceptable salt thereof, and a second therapeutic agent which is selected from the group consisting of: (i) 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (rineterkib or Compound B), or a pharmaceutically acceptable salt thereof, (ii) trametinib (Compound C), or a pharmaceutically acceptable salt or solvate thereof (particularly the DMSO solvate thereof), and (iii) ribociclib (Compound D), or a pharmaceutically acceptable salt thereof, (particularly the succinate salt thereof) for use in the treatment of NRAS-mutant melanoma and/or BRAF-mutant melanoma, especially unresectable or metastatic NRAS-mutant melanoma, and/or unresectable or metastatic BRAF-mutant melanoma, and as further described below.

The invention also provides a pharmaceutical combination which comprises (i) naporafenib and rineterkib; (ii) naporafenib and trametinib; or (iii) naporafenib and ribociclib: where the two compounds are prepared and/or used (or for use) (where the use is as defined above or below) for simultaneous, separate or sequential administration for the treatment of the respective melanoma, and to a pharmaceutical composition comprising such a combination; a method of treating a patient suffering from said melanoma comprising administration of a therapeutically effective amount of the combination or composition to a patient in need thereof; use of such combination or composition for the treatment of said melanoma; and a commercial package comprising such combination, especially for use as defined above or below, and preferably including instructions for such use.

The invention also provides naporafenib, rineterkib, trametinib or ribociclib for use in the treatment of melanoma as described herein, wherein the treatment further comprises administration of the other combination partner in the pharmaceutical combinations of the invention.

### BACKGROUND OF THE INVENTION

Melanoma is the most aggressive form of all skin melanomas. The global incidence of melanoma is approximately 160,000 new cases per year, with 48,000 deaths (Ciurea A. (2016) Epidemiology and Clinical Characteristics of Melanoma. In: Torres-Cabala C., Curry J. (eds) Genetics of Melanoma. Melanoma Genetics. Springer, New York, NY).

The RAS/RAF/MEK/ERK or MAPK pathway is a key signaling cascade that drives cell proliferation, differentiation, and survival. The RAS proteins are a superfamily of GTPases, which include KRAS as well as NRAS and HRAS.

Activation of the MAPK pathway is critical in melanoma. Melanoma can be grouped into molecular subtypes based on their main genetic driver.

The most common mechanism of oncogenic activation of the MAPK pathway in melanoma is of t constitutive activation of the BRAF kinase via mutation, which occurs in ˜40-60% of cases. BRAF encodes a cytoplasmic serine-threonine kinase. More than 97% of BRAF mutations in melanoma are located in codon 600 of the BRAF gene. The V600E mutation encodes a valine to glutamic acid substitution that exposes the active site of BRAF, enabling its constitutive activation as monomers or dimers independent of RAS. In melanoma cells that express wild-type BRAF, or in the normal cells of patients with BRAFV600 driven melanomas, inhibitors such as vemurafenib paradoxically activate RAF signaling.

BRAF (especially BRAFV600) mutation-positive unresectable or metastatic melanomas are aggressive malignancies with poor overall survival. According to the current management of unresectable and/or metastatic melanoma, first-line therapy options include anti-PD-1 monotherapy (pembrolizumab or nivolumab) or combinations thereof, e.g., pembrolizumab and ipilimumab or nivolumab and ipilimumab. For patients with BRAFV600 activating mutations, combined BRAF and MEK inhibitors such as dabrafenib and trametinib, vemurafenib and cobimetinib, and encorafenib and binimetinib are used as first- or second-line therapy. However, patients often progress on these therapies and there are currently no standard therapeutic regimens after progression.

The second most common MAPK pathway aberration in melanoma is mutated NRAS, occurring in ˜15-20% of cases. In melanoma, the majority of activating variants in NRAS occur at codon 61, with variants at codons 12 and 13 occurring less frequently (Gao et al, Sci Signal, 2013; van Elsas, Recent Results Melanoma Res. 1995). NRAS mutant melanoma shows aggressive behavior, with a high rate of liver and brain metastases already present at initial diagnosis (Bergamasco et al. 2016), and, therefore, poor prognosis.

Selective pharmacological inhibition of NRAS remains technically challenging because its GTPase activity has so far eluded the successful design of specific small-molecule antagonists. In addition, there is no established therapies specific for NRAS-mutant melanoma and response to standard of care chemotherapy such as dacarbazine is very limited. A Phase 3 study demonstrated some benefit of the MEK inhibitor binimetinib as compared to standard of care chemotherapy with dacarbazine, e.g. improved overall response rate of 15 vs. 7%. However, discontinuation rate as a result of adverse events suspected to be related to study drug was high (20% vs. 5%), and the benefit in Progression Free Survival (PFS) did not transfer into improvements in overall survival (Dummer et al., Binimetinib versus dacarbazine in patients with advanced NRAS-mutant melanoma (NEMO): a multicentre, open-label, randomised, phase 3 trial; Dummer R., et al.; Lancet Oncology Vol. 18: 4; 435-445: 2017).

### SUMMARY OF THE INVENTION

There is thus a high unmet medical need for patients suffering from NRAS-mutant melanoma and BRAF-mutant melanoma, particularly for NRAS-mutant melanoma. This is particularly true for patients who have received and failed on or progressed on previous therapy for melanoma. Prior therapy may include standard care chemotherapy (e.g., dacarzabine treatment), immunotherapy (e.g., treatment with pembrolizumab, ipilimumab, or nivolumab and combinations thereof), targeted therapy (e.g., treatment with dabrafenib and trametinib: vemurafenib and cobimetinib; and encorafenib and binimetinib). Patients likely to benefit from the combinations of the invention include patients suffering from NRAS-mutant melanoma. BRAF-mutant melanoma, especially where the melanoma is cutaneous melanoma which is unresectable and/or metastatic.

There is also a need for targeted therapy that is safe and/or well tolerated. A therapy which results in durable and sustained responses in a clinical setting would also be beneficial for these patients.

The present invention provides a combination of the invention for use as described herein to treat melanoma in such a patient.

It was found that the combined treatment of naporafenib (Compound A) and a second therapeutic agent which is selected from the group consisting of (i) rineterkib (Compound B), (ii) trametinib (Compound C) and (iii) ribociclib (Compound D) was well tolerated and led to increased anti-tumor response compared to single agents in NRAS-mutant patient derived melanoma xenografts. These data indicate that the combination activity of naporafenib and a second therapeutic agent such as (i) rineterkib, (ii) trametinib and (iii) ribociclib is expected to result in treatment of melanoma which is effective and achievable at clinically tolerable doses. The combination therapy is expected to achieve greater and more durable responses in NRAS mutant melanoma patients.

As the combined administration of Compound A and a second therapeutic agent which is selected from the group consisting of (i) Compound B, (ii) trametinib (Compound C) and (iii) ribociclib (Compound D) was found to suppress oncogenic signaling in the MAPK pathway in such a beneficial manner, it is also likely to bring clinical benefit to BRAF-mutant melanoma patients, since BRAF is a key component of the Ras/Raf/MAPK pathway.

The present invention therefore provides a pharmaceutical combination which comprises the Compound of formula (I) (Compound A, also known as naporafenib),

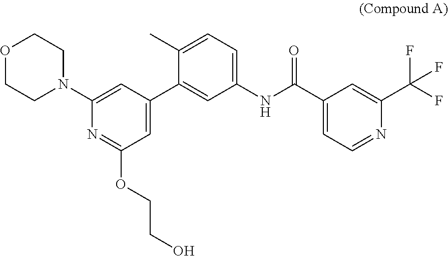 [SMILES: Cc1ccc(NC(=O)c2ccnc(C(F)(F)F)c2)cc1-c1cc(OCCO)nc(N2CCOCC2)c1]

or a pharmaceutically acceptable salt thereof, and a second therapeutic agent which is selected from the group consisting of:

(i) an ERK (especially ERK1/2) inhibitor which is Compound B (also known as rineterkib),

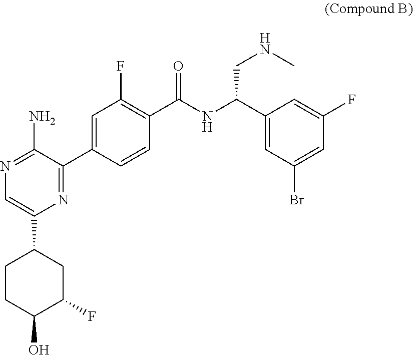 [SMILES: CNC[C@@H](NC(=O)c1ccc(-c2nc([C@H]3CC[C@H](O)[C@@H](F)C3)cnc2N)cc1F)c1cc(F)cc(Br)c1]

or a pharmaceutically acceptable salt thereof, especially the HCl salt;

(ii) a MEK (MEK1/2) inhibitor which is Compound C,

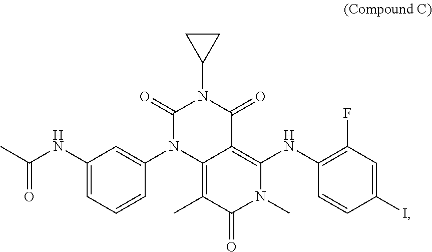 [SMILES: CC(=O)Nc1cccc(-n2c(=O)n(C3CC3)c(=O)c3c(Nc4ccc(I)cc4F)n(C)c(=O)c(C)c32)c1]

(also known as trametinib), or a pharmaceutically acceptable salt or solvate thereof, especially the dimethyl sulfoxide (DMSO) solvate thereof; and

(iii) a CDK4/6 inhibitor which is Compound D,

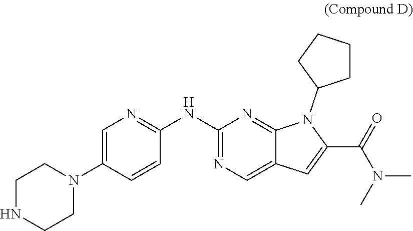 [SMILES: CN(C)C(=O)c1cc2cnc(Nc3ccc(N4CCNCC4)cn3)nc2n1C1CCCC1]

(also known as ribociclib), or a pharmaceutically acceptable salt thereof, especially the succinate salt thereof, for use in the treatment of melanoma as described herein, and in particular, NRAS-mutant melanoma or BRAF-mutant melanoma, especially wherein the melanoma is unresectable, cutaneous melanoma and/or metastatic cutaneous melanoma.

These three dual combinations are also referred herein as a “combination of the invention”. The present invention also provides the Compound of formula (1), or a pharmaceutically acceptable salt thereof, for use in treating NRAS-mutant melanoma and/or BRAF-mutant melanoma, as described herein, by co-administration with a second therapeutic agent which is selected from the group consisting of:
(i) 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound B), or a pharmaceutically acceptable salt thereof (particularly the hydrochloride salt thereof);
(ii) trametinib (Compound C), or a pharmaceutically acceptable salt or solvate thereof (particularly the DMSO solvate thereof), and
(iii) ribociclib (Compound D), or a pharmaceutically acceptable salt thereof, (particularly the succinate salt thereof).

The present invention provides a therapeutic agent which is selected from the group consisting of:
(i) 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound B), or a pharmaceutically acceptable salt thereof (particularly the hydrochloride salt thereof);
(ii) trametinib (Compound C), or a pharmaceutically acceptable salt or solvate thereof (particularly the DMSO solvate thereof), and

(iii) ribociclib (Compound D), or a pharmaceutically acceptable salt thereof, (particularly the succinate salt thereof) for use in treating NRAS-mutant melanoma and/or BRAF-mutant melanoma, and as described herein, by co-administration with the Compound of formula (1), or a pharmaceutically acceptable salt thereof.

The present invention provides a pharmaceutical combination of the invention or a therapeutic agent as described above for use in the treatment of NRAS-mutant melanoma or BRAF-mutant melanoma wherein the combination or the therapeutic agent is administered to the patient and the treatment is accompanied by one or more of the features below:
the patient experiences at least 3 months progression free survival (PFS) after administration of the combination of the present invention;
the patient experiences at least 7 months duration of response (DOS) after administration of the combination of the present invention;
the treatment is accompanied by an increase in an improvement of the overall response rate (ORR), disease control rate (DCR), duration of response (DOR), progression free survival (PFS), or median overall Survival (mOS), particularly as compared to standard of care or other therapy, e.g. v/s binimetinib or v/s dacarbazine administration; and
by a decrease in an adverse effect, optionally wherein the adverse effect is selected from QTc prolongation; an adverse cardiac event and skin toxicity.

In another aspect, the invention provides the use of the pharmaceutical combination of the present invention for the preparation of a medicament for the treatment of NRAS-mutant melanoma and/or BRAF-mutant melanoma, especially wherein the melanoma is unresectable, cutaneous and/or metastatic melanoma, and as described herein.

In another aspect, the invention provides a method for treating a patient suffering from NRAS-mutant melanoma and/or BRAF-mutant melanoma comprising administering to a subject in need thereof a therapeutically effective amount of a pharmaceutical combination the present invention, especially wherein the melanoma is unresectable cutaneous melanoma and/or metastatic cutaneous melanoma, and as described herein.

The present invention also provides a method of treating NRAS-mutant melanoma and/or BRAF-mutant melanoma, as described herein comprising simultaneously, separately or sequentially administering to a subject in need thereof a combination of the invention in a quantity which is jointly therapeutically effective against said melanoma.

The present invention also provides a pharmaceutical composition or combined preparation comprising a quantity of the combination of the invention, which is jointly therapeutically effective against a cancer. and optionally at least one pharmaceutically acceptable carrier for use in the treatment of NRAS-mutant melanoma and/or BRAF-mutant melanoma, and as described herein.

The present invention also provides a combined preparation comprising (a) one or more dosage units of the Compound of formula (I), or a pharmaceutically acceptable salt thereof, and (b) one or more dosage units of the second therapeutic agent thereof, for use in the treatment of NRAS-mutant melanoma or BRAF-mutant melanoma, and as described herein.

The present invention also provides a commercial package comprising as active ingredients a combination of the invention and instructions for simultaneous, separate or sequential administration of a combination of the invention to a patient in need thereof for use in the treatment of a NRAS-mutant melanoma or BRAF-mutant melanoma, as described herein.

The present invention also provides:
Naporafenib for use in combination therapy with trametinib for treating NRAS-mutant melanoma, wherein naporafenib and trametinib is administered to a patient in need, optionally wherein the treatment is accompanied by an increase in an improvement of the overall response rate (ORR), disease control rate (DCR), duration of response (DOR), progression free survival (PFS), or median overall Survival (mOS), e.g. v/s standard of care or other therapy, e.g. v/s binimetinib or v/s dacarzabine; and/or accompanied by a decrease in an adverse effect, optionally wherein the adverse effect is selected from QTc prolongation; an adverse cardiac event and skin toxicity.
A method of improving the overall response rate (ORR), disease control rate (DCR), duration of response (DOR), progression free survival (PFS), or median overall Survival (mOS) in NRAS mutant melanoma or BRAF-mutant melanoma wherein naporafenib is administered in combination with trametinib, or a pharmaceutically acceptable salt or solvate thereof to a patient in need thereof.
A method of improving the overall response rate (ORR), disease control rate (DCR), duration of response (DOR), progression free survival (PFS), or median overall Survival (mOS) in NRAS mutant melanoma or BRAF-mutant melanoma wherein naporafenib is administered in combination with rineterkib, or a pharmaceutically acceptable salt thereof, to a patient in need thereof.
A method of reducing the risk of QTc prolongation and treating a patient suffering from NRAS-mutant or BRAF-mutant melanoma comprising administering to the patient in need thereof a therapeutically effective amount of naporafenib, or a pharmaceutically acceptable salt thereof, and a therapeutically effective amount of trametinib, or a pharmaceutically acceptable solvate thereof.
A method of reducing the risk of QTc prolongation and treating a patient suffering from NRAS-mutant or BRAF-mutant melanoma comprising administering to the patient in need thereof a therapeutically effective amount of naporafenib, or a pharmaceutically acceptable salt thereof, and a therapeutically effective amount of rineterkib, or a pharmaceutically acceptable solvate thereof.

Various aspects of the invention are described in further detail below. Additional definitions are set out throughout the specification.

### BRIEF DESCRIPTION OF THE FIGURES

FIG. 1 is a waterfall plot of responses to Compound A (LXH254) and trametinib across ten patient derived NRAS^{mut} melanoma tumor xenograft models in mice.

FIG. 2 is a Kaplan-Meier plot of time that tumors reached a size of 700 mm³ during daily treatment of single agents Compound A (LXH254), trametinib or combination of both agents.

FIG. 3 is a waterfall plot of responses to Compound A (LXH254) and Compound B across ten patient derived NRAS^{mut} melanoma tumor xenograft models in mice.

FIG. 4 is a Kaplan-Meier plot of time that tumors reached a size of 700 mm³ during daily treatment of single agents Compound A (LXH254), Compound B or combination of both agents.

FIG. 5 is a waterfall plot of responses to Compound A (LXH254) and ribociclib (LEE011) across nine patient derived NRAS^{mut} melanoma tumor xenograft models in mice.

FIG. 6 is a Kaplan-Meier plot of time that tumors reached a size of 700 mm³ during daily treatment of single agents Compound A (LXH254), ribociclib (LEE011) or combination of both agents.

### DETAILED DESCRIPTION OF THE INVENTION

The present invention provides a combination of the invention as described above for use in the treatment of JNRAS-mutant melanoma or BRAF-mutant melanoma, especially wherein said melanoma is unresectable, cutaneous melanoma and/or metastatic cutaneous melanoma. In the present invention, the administration of a combination of the invention is expected to result in a more beneficial effect, e.g., a synergistic or improved anti-proliferative effect, e.g., with regard to the delay of progression or inhibiting the cancer or its symptoms, and may also provide further beneficial effects such as any one or more of the following: lower risk of cardiac adverse events (e.g. lower QTc liability), fewer side-effects such as skin-related toxicity (e.g., rash) and gastro-intestinal-toxicity (e.g. diarrhea), improved tolerability, higher quality of life and decreased morbidity, as compared to standard of care therapy, immunotherapy to targeted monotherapy with any one of the combination partners, therapy with a combination of BRAF and MEK inhibitors or any other previous therapy for melanoma.

There is therefore provided a combination of the present invention for use in the treatment of NRAS-mutant melanoma or BRAF-mutant melanoma, wherein the treatment has a higher clinical efficacy compared to previous treatment, for example as measured by higher confirmed objective response rate (ORR) by local investigator's assessment per RECIST v1.1. Higher clinical efficacy may also be measured by measuring overall response rate (ORR), disease control rate (DCR), duration of response (DOR), progression free survival (PFS) as per RECIST version 1.1 and overall survival (OS). The more beneficial effect may, e.g., be measured by an improved overall response rate obtained with the previous or other therapy.

The therapeutic agents of the combination of the invention may be separately, simultaneously or sequentially administered to a subject in need thereof. Preferably, these therapeutic agents are administered at therapeutically effective dosages which, when combined, provide a beneficial effect. Thus, in one embodiment of the present invention, the combination of the invention is for use in the treatment of NRAS-mutant melanoma or BRAF-mutant melanoma, particularly a melanoma as described herein. The individual components of the dual combinations of the invention are also for use in the simultaneous, separate or sequential administration for the treatment of NRAS-mutant (preferably unresectable and/or metastatic) cutaneous melanoma and/or BRAF-mutant (preferably cutaneous, unresectable and/or metastatic) cutaneous melanoma, especially as defined in the other invention embodiments above and below.

Compound A is Example 1156 in published PCT application WO2014/151616.

Compound A is N-(3-(2-(2-hydroxyethoxy)-6-morpholinopyridin-4-yl)-4-methylphenyl)-2-(trifluoromethyl)isonicotinamide and is the compound of the following structure:

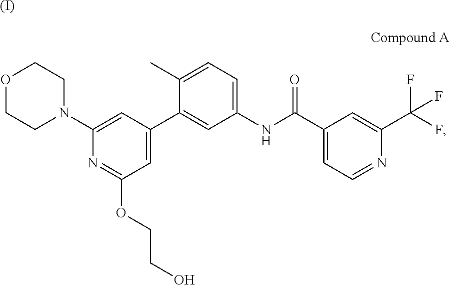 [SMILES: "(I)" ; Cc1ccc(NC(=O)c2ccnc(C(F)(F)F)c2)cc1-c1cc(OCCO)nc(N2CCOCC2)c1]

The preparation of Compound A, pharmaceutically acceptable salts of Compound A and pharmaceutical compositions comprising compound A are also disclosed in the PCT application, e.g., see pages 739-741. Compound A is a selective inhibitor of BRAF and CRAF. Compound A is an adenosine triphosphate (ATP)-competitive inhibitor of BRAF and CRAF protein kinases. Compound A is also known by the code “LXH254” or as “naporafenib”. Throughout the present disclosure, Compound A is also referred to as a CRAF inhibitor or CRAF kinase inhibitor.

Compound B is 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide and is the compound of the following structure.

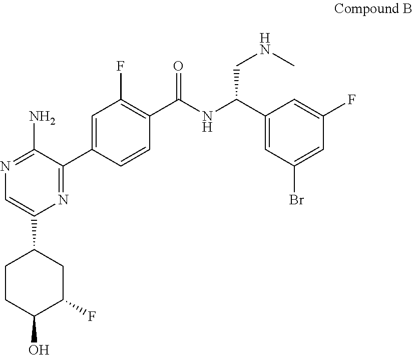 [SMILES: CNC[C@@H](NC(=O)c1ccc(-c2nc([C@H]3CC[C@H](O)[C@@H](F)C3)cnc2N)cc1F)c1cc(F)cc(Br)c1]

Compound B is disclosed and its preparation described in published PCT patent application WO2015/066188. Compound B is an inhibitor of extracellular signal-regulated kinases 1 and 2 (ERK 1/2). Compound B is also known by the code “LTT462” or as “rineterkib”. A particularly useful salt, also for the purposes of the present invention embodiments, of rineterkib is the hydrochloride salt thereof.

Compound C (also known as trametinib) is disclosed and its preparation described e.g., in WO2005/121142, for example in Example 4-1 or in “Example 4-1 (alternative method)” and marketed as Mekinist, trametinib is an approved inhibitor of the MEK1/2 kinases.

Compound C is a potent and selective MEK 1/2 inhibitor. It is N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl)acetamide or trametinib and is the compound having the following structure:

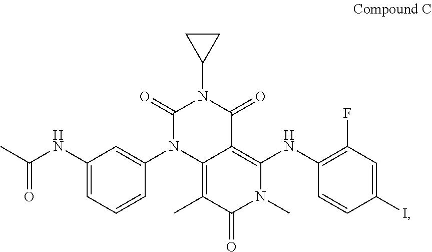 [SMILES: CC(=O)Nc1cccc(-n2c(=O)n(C3CC3)c(=O)c3c(Nc4ccc(I)cc4F)n(C)c(=O)c(C)c32)c1]

A particularly useful solvate, also for the purposes of the present invention embodiments, of trametinib is the dimethyl sulfoxide (DMSO) solvate thereof.

Compound D (also known as ribociclib) is disclosed and its preparation described e.g. in WO 2010/020675, for example in Example 74; the synthesis of the succinate salt is disclosed in US2013/0217698. Ribociclib is an approved selective inhibitor of cyclin DI (CDK4) and CDK6 kinases (a CDK4/6 inhibitor).

The chemical name of ribociclib is 7-cyclopentyl-2-(5-piperazin-1-yl-pyridin-2-ylamino)-7H-pyrrolo[2,3-d]pyrimidine-6-carboxylic acid dimethylamide, and it is the compound with the following structural formula:

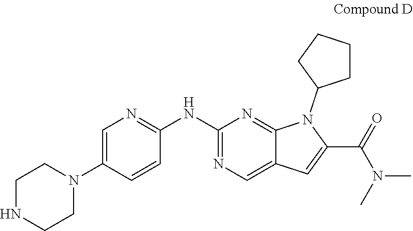 [SMILES: CN(C)C(=O)c1cc2cnc(Nc3ccc(N4CCNCC4)cn3)nc2n1C1CCCC1]

Ribociclib (used under the trademark Kisqali®) is an orally bioavailable and highly selective small molecule inhibitor with highly specific inhibitory activity against CDK4/cyclin-D1 and CDK6/cyclin-D3 enzyme complexes. A particularly useful salt, also for the purposes of the present invention embodiments, of ribociclib is the succinate salt thereof.

Unless otherwise indicated herein or clearly contradicted by context, where reference is made to Compound A, Compound B, Compound C or Compound D, the skilled person will understand that the reference will include the free compounds and/or a pharmaceutically acceptable salt thereof, or, in the case of Compound C, a pharmaceutically solvate, e.g., the dimethyl sulfoxide (DMSO) solvate thereof.

References in this specification to “the invention” are intended to reflect embodiments of the several inventions disclosed in this specification, and should not be taken as unnecessarily limiting of the claimed subject matter.

The following definitions of more general terms or features of the invention can be used to replace one, more than one or all terms features of each invention embodiment, thus resulting in more specific invention embodiments which all form part of the invention.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise. The term “and/or” means that any single one of the features mentioned in connection therewith, any combination of two or three thereof or all features are intended to be included in the definition including “and/or”.

“About” and “approximately” generally mean an acceptable degree of error for the quantity measured given the nature or precision of the measurements. Exemplary degrees of error are within 20 percent (%), typically, within 10%, and more typically, within 5% of a given value or range of values. When describing a dosage or dose herein as a specified amount (i.e., without the term “about” preceding the specified amount, e.g. 200 mg), or as “about” a specified amount (e.g. about 200 mg) the actual dosage or dose can vary by up to 10% from the stated amount: this usage recognizes that the precise amount in a given dosage form may differ slightly from an intended amount for various reasons without materially affecting the in vivo effect of the administered compound.

The skilled person will understand that where a dose or dosage of a therapeutic compound is quoted herein, that amount refers to the amount of the therapeutic compound in its free form. For example, when a dosage of 200 mg (or about 200 mg) of Compound B is referred to, and Compound B is used as its hydrochloride salt. the amount of the therapeutic agent used is equivalent to 200 mg (or about 200 mg) of the free form of Compound B.

The terms “comprising” and “including” are used herein in their open-ended and non-limiting sense unless otherwise noted.

By “a combination” or “in combination with” or “co-administration” or “co-administered with”, it is not intended to imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

The combinations of the invention have therapeutic or protective functions or both.

The terms “combination”, “therapeutic combination” or “pharmaceutical combination” as used herein refer to either a fixed combination in one dosage unit form, or non-fixed combination, or a kit of parts for the combined administration (co-administration) where two or more therapeutic agents may be administered together, independently at the same time or separately within time intervals, especially where these time intervals allow that the combination partners show a cooperative, e.g., synergistic, effect.

The combination therapy or the method of treatment of melanoma described herein refers to the administration of two or more therapeutic agents to treat a melanoma as described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration and co-administration also encompass use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

By simultaneous therapeutic use, within the meaning of the present invention is meant an administration of at least two active ingredients by the same route and at the same time or at substantially the same time.

By separate use, within the meaning of the present invention is meant in particular an administration of at least two active ingredients at the same time or at substantially the same time by different routes.

By sequential therapeutic use is meant administration of at least two active ingredients at different times, the administration route being identical or different. More particularly by an administration method is meant according to which the whole administration of one of the active ingredients is carried out before administration of the other or others commences.

The terms “fixed combination”, “fixed dose” and “single formulation” as used herein refers to a single carrier or vehicle or dosage form formulated to deliver an amount, which is jointly therapeutically effective for the treatment of cancer, of both therapeutic agents to a patient. The single vehicle is designed to deliver an amount of each of the agents along with any pharmaceutically acceptable carriers or excipients. In some embodiments, the vehicle is a tablet, capsule, pill, or a patch. In other embodiments, the vehicle is a solution or a suspension.

The term “non-fixed combination” or “kit of parts” means that the therapeutic agents of combination of the invention are both administered to a patient as separate entities either simultaneously, concurrently or sequentially with no specific time limits, wherein such administration provides therapeutically effective levels of the two compounds in the body of a subject in need thereof. The latter also applies to cocktail therapy, e.g., the administration of three or more active ingredients.

The term “pharmaceutically acceptable” as used herein refers to those compounds, materials, compositions and/or dosage forms, which are, within the scope of sound medical judgment, suitable for contact with the tissues of a subject, e.g., a mammal or human, without excessive toxicity, irritation, allergic response and other problems or complications commensurate with a reasonable benefit/risk ratio.

As used herein, the term “pharmaceutically acceptable excipient” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art. Except insofar as any conventional carrier is incompatible with the active ingredient, its use in the therapeutic or pharmaceutical compositions is contemplated.

The term “pharmaceutical composition” is defined herein to refer to a mixture or solution containing at least one therapeutic agent to be administered to a patient in order or treat a particular disease or condition affecting the subject. The present pharmaceutical combinations can be formulated in suitable pharmaceutical compositions for enteral or parenteral administration, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. The pharmaceutical composition may contain, from about 0.1% to about 99.9%, preferably from about 1% to about 60%, of the therapeutic agent(s). One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms: The Handbook of Pharmaceutical Excipients, Rowe et al., Eds., American Pharmaceuticals Association; and Remington: the Science and Practice of Pharmacy, Gennaro, Ed., Lippincott Williams & Wilkins. These optional additional conventional carriers may be incorporated into the oral dosage form either by incorporating the one or more conventional carriers into the initial mixture before or during granulation or by combining one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Pharmaceutical compositions may be presented in unit dose forms containing a predetermined amount of active ingredient per unit dose. In certain embodiments, the unit dose includes one or more vehicles such that each vehicle includes an effective amount of at least one of the therapeutic agents along with pharmaceutically acceptable carriers and excipients. In some embodiments, the unit dose is one or more tablets, capsules, pills, injections, infusions, patches, or the like, administered to the patient at the same time. As is known to those skilled in the art, the amount of active ingredient per dose will depend on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art.

The pharmaceutical compositions of the invention may include a “therapeutically effective amount” or “effective amount” of a compound of the invention. The term “pharmaceutically effective amount”, “therapeutically effective amount” or “clinically effective amount” of a combination of therapeutic agents is an amount sufficient, at dosages and for periods of time necessary, to provide an observable or clinically significant improvement over the baseline of clinically observable signs and symptoms of the disorders treated with the combination. A therapeutically effective amount vary according to factors such as the disease state, age, sex, and weight of the individual. A therapeutically effective amount is also one in which any toxic or detrimental effects of the therapeutic agents are outweighed by therapeutically beneficial effects. A “therapeutically effective dosage” preferably modulates a measurable parameter, such as tumor growth rate or disease progression in a desired manner. The ability of a compound to modulate a measurable parameter can be evaluated in an animal model system predictive of efficacy in human tumors to help establish suitable dosing levels and schedules. Alternatively, this property of a composition can be evaluated by examining the ability of the compound to modulate an undesired parameter by using in vitro assays known to the skilled practitioner.

The term “jointly therapeutically active” or “joint therapeutic effect” as used herein means that the therapeutic agents can be given jointly, separately or sequentially in such time intervals that they prefer such that the subject, especially human, to be treated, still show an(preferably synergistic) interaction (joint therapeutic effect). Whether this is the case can, inter alia, be determined by following the blood levels of the compounds, showing that both compounds are present in the blood of the human to be treated at least during certain time intervals.

An “oral dosage form” includes a unit dosage form prescribed or intended for oral administration.

Each of Compound. A, Compound B, Compound C and Compound D may be administered in an oral dosage form.

As used herein, the terns “treat”, “treatment” and “treating” refer to the reduction or amelioration of the progression, severity and/or duration of a disorder, e.g., a proliferative disorder, or the amelioration of one or more symptoms, suitably of one or more discernible symptoms, of the disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments, the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The terms “treat”, “treatment” and “treating” include the reduction of the incidence and severity of adverse events (AEs) and serious AEs (SAEs) including changes in laboratory values, vital signs and Electrocardiograms (ECGs) in a patient or a patient population.

The terms “treat”, “treatment” and “treating” include an improvement of the overall response rate (ORR), disease control rate (DCR), duration of response (DOR), or progression free survival (PFS), e.g., as per Response Evaluation Criteria in Solid Tumors (RECIST) version 1.1, in a patient or a patient population.

Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent, delay, or treat, or all, as appropriate, development, continuance or aggravation of a disease in a subject, e.g., a mammal or human.

The present invention therefore provides a combination of the invention for use in the treatment of BRAF-mutant and/or NRAS-mutant melanoma in a patient wherein the treatment is accompanied by an increase in an improvement of the overall response rate (ORR), disease control rate (DCR), duration of response (DOR), progression free survival (PFS), or median overall survival (mOS) e.g. v/s standard of care or other therapy, e.g. v/s binimetinib or v/s dacarzabine.

| NEMO | Binimetinib | Dacarbazine |
|---|---|---|
| ORR | 15% | 7% |
| PFS | 2.8 months | 1.5 months |
| DOR | 6.9 months | Not estimable |
| mOS | 11 months | 10.1 months |

The term “patient” as used herein is intended to include animals, but is preferably a human patient. The patient is especially a human patient in need of melanoma treatment. For example, the patient is a patient suffering from late stage melanoma, or metastatic melanoma, or unresectable melanoma. In some instances, the human patient has received and progressed on prior therapy with another agent. The melanoma may be NRAS-mutant, or BRAT-mutant or NRAS-mutant melanoma, as described herein.

The term “inhibition” or “inhibitor” includes a reduction in a certain parameter, e.g., an activity, of a given molecule or pathway. For example, inhibition of an activity of a targeted kinase (Raf or CDK4/6) by 5%, 10%, 20%, 30%, 40% or more is included by this term. Thus, inhibition may be, but need not be, 100%.

As used herein, “salts” (which, what is meant by “or salts thereof” or “or a salt thereof”), can be present alone or in mixture with free compounds of the combination of the invention, e.g., Raf inhibitor Compound of formula (1) or CDK4/6 inhibitor, preferably ribociclib, and are preferably pharmaceutically acceptable salts. Such salts are formed, for example, as acid addition salts, preferably with organic or inorganic acids, from compounds of the combination of the invention with a basic nitrogen atom, especially the pharmaceutically acceptable salts. The term “pharmaceutically acceptable salts” refers to salts that retain the biological effectiveness and properties of the compound and which typically are not biologically or otherwise undesirable.

Lists of suitable salts can be found, in “Remington's Pharmaceutical Sciences” and “Remington: the science and practice of pharmacy”.

The term “synergistic effect” as used herein, refers to action of two agents such as, for example, Raf inhibitor Compound with formula (I), or a pharmaceutically acceptable salt thereof, and a second therapeutic agent selected from Compound B, Compound C and Compound D, or a pharmaceutically acceptable salt thereof (or in the case of Compound C, a pharmaceutically acceptable solvate thereof), to produce an effect, for example, slowing the symptomatic progression of cancer or symptoms thereof, which is greater than the simple addition of the effects of each drug administered by themselves.

Where reference is made to the Overall Response Rate (ORR), this (if not defined otherwise at a specific position of this specification) refers to the sum of Partial Response and Complete Response (defined as PR+CR (Partial Response plus Complete Response) according to the Response Evaluation Criteria In Solid Tumors (RECIST v1.1). Tumor evaluations and assessment of tumor burden can be made based on RECIST criteria (Therasse et al 2000), New Guidelines to Evaluate the Response to Treatment in Solid Tumors, Journal of National Melanoma Institute, Vol. 92; 205-16 and is made within the present invention according to the revised RECIST guidelines (version 1.1) (Eisenhauer et al 2009, New response evaluation criteria in solid tumours: revised RECIST guideline (version 1.1), Eur J Melanoma, 45(2): 228-47.). In order to avoid clarity issues, where the ORR is defined herein as at least 15% or as at least 30%, the upper limit of the ORR can be 100%. Ili specific embodiments, the ORR is any one of 30, 35, 40, 45, 50, 55, 60, 65, 70, 75 or 80%, (the higher the value, the more 23 preferred) or higher.

Complete Response is defined as disappearance of all non-nodal target lesions. In addition, any pathological lymph nodes assigned as target lesions must have a reduction in short axis to <10 mm 1.

Partial Response is defined as at least a 30% decrease in the sum of diameter of all target lesions, taking as reference the baseline sum of diameters.

Other response criteria can be deduced from the following table (according to RECIST v1.1):

| Response Criteria | Evaluation of target lesions |
|---|---|
| Progressive Disease (PD): | At least a 20% increase in the sum of diameter of all measured target |
| | lesions, taking as reference the smallest sum of diameter of all target |
| | lesions recorded at or after baseline. In addition to the relative increase of |
| | 20%, the sum must also demonstrate an absolute increase of at least 5 mm². |
| Stable Disease (SD): | Neither sufficient shrinkage to qualify for PR or CR nor an increase in |
| | lesions which would qualify for PD. |
| Unknown (UNK) | Progression has not been documented and one or more target lesions have |
| | not been assessed or have been assessed using a different method than |
| | baseline. |

The progression of melanoma, tumor burden increase or decrease, and response to treatment with an inhibitor combination according to the invention may be monitored by methods well known to those in the art. Thus the progression and the response to treatment may be monitored by way of visual inspection of the melanoma, such as, by means of X-ray, CT scan or MRI or by tumor biomarker detection.

The QT interval is the time interval between the start of the Q wave and the end of the T wave in the cardiac cycle. The term “QTc” refers to a QT interval which is corrected for heart rate and takes into account the physiologic shortening of the QT interval which occurs as the heart rate increases. This correction permits comparison of the QT interval across a range of rates. A prolonged QTc is associated with an increased risk of sudden cardiac death. Treatment of any disease condition should therefore seek to minimize the risk of QTc prolongation and lower the QTc liability of the proposed treatment.

The term QTcF refers to the QT interval corrected by Fridericia's formula

Changes and increases in QTc and QTcF may be measured by performing an electrocardiogram (ECG).

It has been found that it is possible to achieve a balance between therapeutic efficacy and the reduction of adverse side-effects (e.g. the reduction of QTc prolongation) with the dosing regimens disclosed herein.

There is thus provided a method of reducing the risk of QTc prolongation in a patient and treating a patient suffering from NRAS-mutant or BRAF-mutant melanoma comprising administering to the patient in need thereof a therapeutically effective amount of naporafenib, or a pharmaceutically acceptable salt thereof, and a therapeutically effective amount of trametinib, or a pharmaceutically acceptable solvate thereof.

Also provided herein is a method of reducing the risk of QTc prolongation and treating a patient suffering from NRAS-mutant or BRAF-mutant melanoma comprising administering to the patient in need thereof a therapeutically effective amount of naporafenib, or a pharmaceutically acceptable salt thereof, and a therapeutically effective amount of rineterkib, or a pharmaceutically acceptable solvate thereof.

In this context, a therapeutically effective amount of naporafenib in particular is less than 800 mg total daily dose, e.g. less than or equal to 400 in total daily dose. Preferably that total daily dose is administered twice a day.

Other therapeutically effective amounts of naporafenib, trametinib and rineterkib are described below and in the Examples.

The combination of the invention may be particularly useful in treating NRAS-mutant melanoma, in particular wherein said melanoma is previously treated unresectable or metastatic NRAS mutant cutaneous melanoma.

NRAS mutations are found in 15%-20% of cutaneous metastatic melanoma in either exon 2 (codon 12 or 13) or exon 3 (codon 61), The NRAS-mutant melanoma to be treated by a combination of the invention therefore includes melanoma with a mutation in NRAS at codon 61, codon 12 or codon 13, especially at codon 61.

NRAS mutations of interest may be selected from G12C, G12R, G12D, G12V, G12S, G12A, G13R, G13D, G13C, G13A, G13, G13S, G13V, Q61R, Q61L, Q61K, Q61H, Q61P, Q61E and combinations thereof. Mutations of interest include G12D or G13D. The term “NRAS-mutant melanoma” includes any melanoma tumor that exhibits a mutated NRAS protein, in particular gain of function NRAS-mutation; especially any G13R, Q61K, Q61L or Q61R NRAS-mutant tumor. Thus NRAS-mutant melanoma includes melanoma having at least one NRAS mutation corresponding to Q61R, Q61L, Q61K, Q61H, Q61P or Q61E, more preferably corresponding to Q61K, Q61L or Q61R. The melanoma to be treated may be NRAS QG13R-mutant melanoma. The NRAS-mutant cutaneous melanoma may be at an early, intermediate or late stage. The NRAS-mutant cutaneous melanoma may be locally advanced or metastatic. The NRAS-mutant cutaneous melanoma may be unresectable.

The combination of the invention may be also useful in treating BRAT'-mutant melanoma, in particular wherein said melanoma is previously treated unresectable or metastatic BRAF-mutant melanoma.

The combination of the invention may be especially useful in treating melanoma with a mutation in BRAF at codon 600.

Most of the BRAE mutations are clustered to two regions: the glycine-rich P loop of the N lobe and the activation segment and flanking regions. V600E mutation has been detected in a variety of cancers, and is due to a substitution of thymine with adenine at nucleotide 1799. This leads to valine (V) being substituted for by glutamate (F) at codon 600 (now referred to as V600E).

BRAF-mutantmelanoma is, for example, melanoma exhibiting a BRAFV600 mutation. A BRAE mutation in melanoma may be selected from BRAF V600E, V600K, V600R, V600R, V600M, V600D, V600G and combinations thereof. A BRAE-mutation is especially V600D, V600E, V600R, V600K, or BRAFD287H mutation. Preferably the term “BRAE-mutant melanoma” refers to BRAFV600E-mutant, BRAFV600K-mutant, BRAFV600R-mutant and BRAFV600D-mutant melanoma, more preferably to BRAFV600E-mutant and BRAFV600D-mutant melanoma, most preferably to BRAFV600E-mutant melanoma. The BRAE-mutant melanoma may be at an early, intermediate or late stage. The BRAE-mutant melanoma may be locally advanced or metastatic. The BRAE-mutant melanoma may be unresectable.

Where “NRAS-mutant melanoma” or “BRAE-mutant melanoma” is mentioned, this relates especially to ocular or cutaneous melanoma—unless context clearly dictates otherwise. Preferably this term refers to cutaneous melanoma, in particular to unresectable and/or metastatic cutaneous melanoma.

The present invention provides a pharmaceutical combination for use in treating melanoma in a patient wherein the melanoma has been previously treated, e.g. by surgical removal, or other therapy and progressed after such therapy.

The melanoma to be treated by the combination may be melanoma which is refractory or resistant to previous treatment with another therapy. The combination of the invention may therefore be useful as second-line, third-line or fourth-line treatment of melanoma.

Prior treatment for melanoma patients includes:
treatment with talimogene laherparepvec (also known by the tradename T-Vec, Imlygic or Oncovex) which is a biopharmaceutical drug to treat unresectable melanoma and which is injected directly into a subset of metastatic lesions:
standard care chemotherapy (e.g., dacarzabine);
treatment with a cytotoxic agent such as a nitrosurea and/or mitomycin C;
immunotherapy (e.g., pembrolizurnab, ipilimumab, or nivolurnab and combinations thereof);
targeted therapy (e.g., dabrafenib and trametinib, vemurafenib and cobimetinib, and encorafenib and binimetinib).

The patient to be treated thus includes a patient suffering from NRAS-mutant melanoma and/or BRAF-mutant melanoma, especially a patient who has received previous therapy including standard care chemotherapy (e.g., dacarzabine), immunotherapy pembrolizumab, ipilimumab, or nivolumab and combinations thereof), targeted therapy (e.g., dabrafenib and trametinib, vemurafenib and cobimetinib, and encorafenib and binimetinib) and who has progressed on previous therapy. The patient may be a patient suffering from NRAS-mutant melanoma and/or BRAF-mutant melanoma especially where the melanoma is cutaneous melanoma which is unresectable and/or metastatic.

In another embodiment, the NRAS-mutant melanoma or BRAF-mutant melanoma is resistant or refractory to standard of care.

In another embodiment, the NRAS-mutant melanoma or BRAF-mutant melanoma is resistant or refractory to standard of care with dacarbazine, preferably when the melanoma is NRAS-mutant melanoma.

In another embodiment, the melanoma is resistant or refractory to treatment with a BRAF inhibitor and/or a MEK inhibitor (i.e. treatment with “RAF+/−MEK inhibitors), preferably when the melanoma is BRAF-mutant melanoma. The BRAF inhibitor may be selected from dabrafenib, vemurafenib and encorafenib. The MEK inhibitor may be selected from trametinib, cobimetinib and binimetinib.

For example, patients likely to benefit from combination therapy as described herein include patients suffering from BRAFV600-mutant melanoma which has been previously treated with one or more of (i) dabrafenib and trametinib, vemurafenib and cobimetinib, and (iii) encorafenib and binimetinib.

In another embodiment, the melanoma is resistant or refractory to treatment with a cytotoxic agent such as a nitrosurea and/or mitomycin C.

In another embodiment, the melanoma is resistant or refractory to treatment with immunotherapy treatment including therapy with one or more immune checkpoint inhibitors.

Thus in one embodiment, the melanoma to be treated by a combination of the present invention is BRAF-mutant melanoma or NRAS-mutant melanoma which is resistant to immunotherapeutic PD-1 (Programmed Cell death 1 receptor) or PD-L1 (the ligand of PD-1) treatment, alone or in combination with an anti-CTLA4 (cytotoxic T-lymphocyte-associated protein) antibody (e.g. See e.g. Tsai et al, Human Vaccines & Immunotherapeutics 10: 11, 3111-3116; November 2014.

Thus in one embodiment, the melanoma to be treated is resistant or refractory to treatment with one or more therapeutic agents selected from ipilimumab, spartalizumab, nivolumab, pembrolizumab, pidizilumab, BMS-9365559, MEDI4736, and MSB0010718C.

Thus, the melanoma to be treated by the combination of the present invention includes BRAF-mutant melanoma or NRAS-mutant melanoma which is resistant to anti-PD-1 monotherapy (such as pembrolizumab or nivolumab) or a combination of anti-PD-1 agent with ipilimumab.

Genetic assessment of BRAT, NRAS and NF1-mutations in patients can be conducted according to methods known in the art, e.g., using SNaPshot or DFCI Oncopanel as described previously (Sholl L M, et al. JCI Insight 2016; 1: e87062; Zheng Z, et al., Nat Med 2014; 20: 1479-84). The current iterations of both assays utilize next-generation sequencing, whereas earlier versions of SNaPshot relied on multiplex PCR. The current version of SNaPshot interrogates exons 11 and 15 of BRAF, exons 2-5 of KRAS and NRAS, and exons 1-58 of NF1. Oncopanel detects alterations involving all exons of BRAF, KRAS, NRAS, and NF1.

Activity of the present combination against melanoma can be experimentally evidenced in vitro in melanoma cell lines such as SKMEL-30 cells (harboring a BRAFD287H mutation).

Alternatively, human tumor xenograft models using mutant melanoma cells can be used, e.g. as described in or analogously to the Examples.

In one embodiment, the invention provides a method of treating; (e.g., inhibiting, reducing, ameliorating, or preventing) BRAF-mutant or NRAS-mutant melanoma in a patient, comprising administering an (or a preferably therapeutically effective) amount of a combination of the invention to the patient in need thereof in a treatment as described herein above. Suitable dosages and administration schedules for using these compounds in such methods are described herein. In particular, therapeutically effective amounts of naporafenib, rineterkib, trametinib and ribociclib, and dosing regimens to be used according to the present invention are to be found in the paragraphs below and in the Examples.

Compound A, Compound B, Compound C or Compound D, or their respective pharmaceutically acceptable salt thereof, is or is to be preferably administered orally.

Compound A, or a pharmaceutically acceptable salt thereof, may be administered continuously, Compound B, or a pharmaceutically acceptable salt thereof, may be administered continuously. Compound C, or its pharmaceutically acceptable salt or solvate thereof, may be administered continuously or intermittently. Ribociclib, or a pharmaceutically acceptable salt thereof, may be administered continuously or intermittently, e.g., administered three weeks on and one week off.

The total daily dose of Compound A, or a pharmaceutically acceptable salt thereof, may be administered once daily, or may be divided into two and each dose of Compound A administered twice daily. Preferably, the total daily dose of the Compound of formula (I) is administered once daily. In another preferred embodiment, the total daily dose of the Compound of formula (I) is administered twice daily.

In the combinations of the invention, Compound A may be administered at a total daily dosage (TTD) from about 50 to about 1200 mg. Thus the combination therapy typically uses 50 mg, 100 mg, 200 mg, 250 mg, 300 mg, 350 mg, 400 mg, 600 mg or 1200 mg total daily dosage (TTD) of Compound A. The TTD may be administered either QD (“qd” or once daily) or BID (“bid” or twice daily). Suitably, in the combinations and methods of the invention, a dosage of 50 mg, 100 mg, 200 mg, 250 mg, 300 or 350 mg of Compound A is administered once daily or a dosage of 100 mg, 200 mg, 300 mg, 400 mg or 600 mg is administered BID. Preferably the TTD dosage of Compound A in the combinations of the present invention is from about 100 mg to 400 mg, e.g. 100 mg, 200 mg or 400 mg, which may be administered on a once daily or twice daily basis.

In an embodiment, Compound A is administered at a TTD of 800 mg. For example, Compound A may be administered a dose of 400 mg, twice a day, or 800 mg once a day.

In a preferred embodiment, Compound A is administered at a TTD of less than 800 mg, such as less than or equal to 400 mg.

In a specially preferred embodiment, Compound A is administered at a TTD of 400 mg, administered twice a day (i.e. 200 mg BID). This dose and dosing regimen for Compound A are expected to provide the optimum balance of efficacy and safety (e.g. lower risk of adverse side-effects such as lower risk of QTcF prolongation whilst maintaining optimum anti-tumor response).

Compound Bas the additional therapeutic agent in the combination according to the present invention is administered to a subject in need thereof in a therapeutically effective amount.

In a preferred embodiment, the total daily dose of Compound B, or a pharmaceutically acceptable salt thereof, is an amount which is selected from about 50 mg to about 300 mg per day; suitably, the amount is selected from about 100 mg to about 200 mg per day. In a preferred embodiment, Compound B, or a pharmaceutically acceptable salt thereof, is administered at a total daily dose which is about 100 or about 200 mg. Alternatively, the total daily dose may be divided in two doses, which are administered twice daily.

As part of the combination therapy of the present invention, Compound B is preferably administered continuously. In particular, the following daily doses may be envisaged as follows:

| Total daily dose of Compound A^{@}- | Total daily dose of Compound B^{b} - |
|---|---|
| preferably administered | preferably administered |
| twice daily | once daily |
| 200 mg | 100 mg, 150 mg or 200 mg; |
| 400 mg | 100 mg, 150 mg or 200 mg; |
| 800 mg | 100 mg, 150 mg or 200 mg; |

@: total daily dose of Compound A may be administered once daily or twice daily, preferably divided up in two equal doses administered twice daily. Thus a total daily dose of 200 mg may be administered in one dose of 200 mg once daily or a dose of 100 mg twice daily

^{b}b: total daily dose of Compound B may be administered once daily or twice daily, preferably once daily

In one embodiment, Compound A is administered at a total daily dose of 800 ng and Compound B is administered at a total daily dose of 200 mg.

In one embodiment, Compound A is administered at a total daily dose of 800 mg and Compound B is administered at a total daily dose of 100 mg.

In one embodiment, Compound A is administered at a total daily dose of 400 mg and Compound B is administered at a total daily dose of 100 mg or 200 mg.

In one embodiment, Compound A is administered at a dose of 200 mg twice a day and Compound B is administered at a dose of 100 mg or 200 mg once a day.

Trametinib as the additional therapeutic agent in a combination according to the present invention is administered to a subject in need thereof in a therapeutically effective amount.

In a preferred embodiment, the total daily dose of trametinib, or a pharmaceutically acceptable salt or solvate thereof, is an amount selected from about 0.5 mg to about 2 mg per day; suitably, the amount is selected from about 0.5, about 1 and about 2 mg of trametinib per day. In a preferred embodiment, trametinib, or a pharmaceutically acceptable salt or solvate thereof, is administered at a total daily dose of 0.5 or 1 mg. Alternatively, the total daily dose may be divided in two doses, which are administered twice daily.

The total daily dose of Compound A may be preferentially administered twice daily, whilst the daily dose of trametinib may be administered once daily. Other doses such as in the Table below may also be administered.

| Total daily dose of Compound A^{@}- | Total daily dose of trametinib^{b} - |
|---|---|
| preferably administered once | preferably administered |
| or twice daily | once daily |
| 200 mg | 0.5 mg, 1.0 mg, 1.5 mg or 2.0 mg |
| 400 mg | 0.5 mg, 1.0 mg, 1.5 mg or 2.0 mg |

| Total daily dose of Compound A^{@}- | |
|---|---|
| preferably administered once or | Total daily dose of trametinib^{b} - |
| twice daily | preferably administered once daily |
| 600 mg | 0.5 mg, 1.0 mg, 1.5 mg or 2.0 mg |
| 800 mg | 0.5 mg, 1.0 mg, 1.5 mg or 2.0 mg |
| 1200 mg | 0.5 mg, 1.0 mg, 1.5 mg or 2.0 mg |

More preferably, the total daily doses may be envisaged as follows:

| Total daily dose of Compound A^{@}- | Total daily dose of trametinib^{b} - |
|---|---|
| preferably administered twice daily | preferably administered once daily |
| 200 mg | 0.5 mg or 1.0 mg |
| 400 mg | 0.5 mg or 1.0 mg |
| 800 mg | 0.5 mg, or 1.0 mg |

@: total daily dose of Compound A may be administered once daily or twice daily, preferably divided up in two equal doses administered twice daily. Thus a total daily dose of 200 mg of Compound A may be administered in one dose of 200 mg once daily or a dose of 100 mg twice daily

^{b}: total daily dose of trametinib may be administered once daily or twice daily, preferably once daily

In a preferred embodiment, Compound A is administered at a total daily dose of 400 mg and trametinib is administered at a total daily dose of 1 mg, in particular to provide the optimum balance of efficacy and safety (e.g. lower risk of adverse side-effects such as lower risk of QTcF prolongation whilst maintaining optimum anti-tumor response).

In a preferred embodiment, Compound A is administered at a dose of 200 mg twice daily and trametinib is administered at a dose of 1 mg once daily, in particular to provide the optimum balance of efficacy and safety (e.g. lower risk of adverse side-effects such as lower risk of QTcF prolongation whilst maintaining optimum anti-tumor response).

Ribociclib as the additional therapeutic agent in the combination according to the present invention is administered to a subject in need thereof in a therapeutically effective amount.

In a preferred embodiment, the total daily dose of ribociclib, or a pharmaceutically acceptable salt thereof, is a an amount which is selected from about 100 mg to about 600 mg per day; suitably, the amount is selected from about 200 mg to about 600 mg per day. In a preferred embodiment, ribociclib, or a pharmaceutically acceptable salt thereof, is administered at a total daily dose which is selected from about 100 mg, about 200 mg, about 400 mg and about 600 mg. Alternatively, the total dose may be divided in two doses, which are administered twice daily.

As part of the combination therapy of the present invention, ribociclib is administered continuously or with a drug holiday period. Thus, ribociclib may be administered three weeks on and one week off. Typically, the total daily dose of ribociclib, (e.g., 400 mg or 600 mg (for instance taken as two or three 200 mg tablets) may be taken (e.g., once daily) for the first 21 consecutive days, followed by a ribociclib drug holiday period of 7 consecutive days. The 28-day cycle is then repeated.

In particular, the following daily doses may be envisaged as follows:

| Total daily dose of | |
|---|---|
| Compound A^{@}- | |
| preferably administered | Total daily dose of ribociclib^{b#} - |
| once or twice daily | preferably administered once daily |
| 200 mg | 100 mg, 200 mg, 400 mg, 600 mg, or 900 mg |
| 400 mg | 100 mg, 200 mg, 400 mg, 600 mg, or 900 mg |
| 600 mg | 100 mg, 200 mg, 400 mg, 600 mg, or 900 mg |
| 800 mg | 100 mg, 200 mg, 400 mg, 600 mg, or 900 mg |
| 1200 mg | 100 mg, 200 mg, 400 mg, 600 mg, or 900 mg |
| 1600 mg | 100 mg, 200 mg, 400 mg, 600 mg, or 900 mg |

More preferably, the total daily doses may be envisaged as follows:

| Total daily dose of | |
|---|---|
| Compound A^{@}- | |
| preferably administered | Total daily dose of ribociclib^{b#} - |
| twice daily | preferably administered once daily |
| 200 mg | 100 mg, 200 mg, 400 mg or 600 mg; |
| | preferably 100 mg, 200 mg or 400 mg; |
| | more preferably 200 mg or 400 mg |
| 400 mg | 100 mg, 200 mg, 400 mg or 600 mg; |
| | preferably 100 mg, 200 mg or 400 mg; |
| | more preferably 200 mg or 400 mg |
| 800 mg | 100 mg, 200 mg, 400 mg or 600 mg; |
| | preferably 100 mg, 200 mg or 400 mg; |
| | more preferably 200 mg or 400 mg |

^{@}: total daily dose of Compound A may be administered once daily or twice daily, preferably divided up in two equal doses administered twice daily. Thus a total daily dose of 200 mg daily may be administered in one dose of 200 mg once daily or a dose of 100 mg twice daily
^{b}: total daily dose of ribociclib may be administered once daily or twice daily, preferably once daily
^{#}: ribociclib is preferably administered three weeks on and one week off

In one embodiment, Compound A is administered at a total daily dose of 800 mg and ribociclib is administered at a total daily dose of 200 mg, optionally wherein Compound A is administered continuously and ribociclib is administered three weeks on and one week off.

In one embodiment, Compound A is administered at a total daily dose of 800 mg and ribociclib is administered at a total daily dose of 400 mg, optionally wherein Compound A is administered continuously and ribociclib is administered three weeks on and one week off.

In one embodiment, Compound A is administered at a dose of 400 mg twice a day and ribociclib is administered at a dose of 200 mg once a day, optionally wherein Compound A is administered continuously and ribociclib is administered three weeks on and one week off.

In one embodiment, Compound A is administered at a dose of 400 mg twice a day and ribociclib is administered at a dose of 400 mg once a day, optionally wherein Compound A is administered continuously and ribociclib is administered three weeks on and one week off.

In one preferred embodiment, Compound A is administered at a total daily dose of 400 mg and ribociclib is administered at a total daily dose of 400 mg, optionally wherein Compound A is administered continuously and ribociclib is administered three weeks on and one week off, in particular to provide the optimum balance of efficacy and safety (e.g. lower risk of adverse side-effects such as lower risk of QTcF prolongation whilst maintaining optimum anti-tumor response).

In one preferred embodiment, Compound A is administered at a dose of 200 mg twice a day and ribociclib is administered at a dose of 400 mg once a day, optionally wherein Compound A is administered continuously and ribociclib is administered three weeks on and one week off, in particular to provide the optimum balance of efficacy and safety (e.g. lower risk of adverse side-effects such as lower risk of QTcF prolongation whilst maintaining optimum anti-tumor response).

Where dosages are given, they may be provided in one unit dosage form, e.g. a tablet or capsule, separately for each of the active ingredients (Compound A, Compound B, Compound C, or Compound D) or with two of them in combination, and/or divided up in 2, 3, 4 or more, e.g. 2 or 3 dosage units.

Especially, treatment with a combination according to the invention is indicated for treatment of such melanoma that does not or does not sufficiently respond to treatment with Compound A alone or Compound B alone or Compound C alone or Compound D alone.

Typically, all compounds used in a combination therapy according to the invention are administered orally.

The combinations as described herein can be administered to the patient systemically (e.g., orally (preferred), parenterally, subcutaneously, intravenously, rectally, intramuscularly, intraperitoneally, intranasally, transdermally), or topically. The combination partners in the dual combination may be administered orally, and may be administered together (at the same time) or separately in any order, following dosing schedules determined by a treating physician; suitable doses and dosing schedules are disclosed herein.

### EXAMPLES

The Examples below are set forth to aid in the understanding of the invention and, though also being specific invention embodiments, are not intended to, and should not be construed to, limit its scope in any way.

### Example 1: Combination Efficacy of Compound A (Also Known as LXH254) and Compound C (Trametinib) in SKMEL-30 Human Tumor Xenografts in Mice

### Methods

### a) Animals and Maintenance Conditions

Outbred athymic (Crl:NU(NCr)-Foxn1^{nu}) female mice (Charles River Lab), weight 19-24 g, age 8 weeks, were allowed to acclimate in the Novartis NIBRI animal facility with access to food and water ad libitum for minimum of 3 days prior to manipulation. The animals were handled in accordance with Novartis ACUC regulations and guidelines.

### b) Drug Formulations

Compound A was dosed p.o. (orally) as a suspension in distilled water made, fresh before every dose by diluting a formulated stock of 50 mg/mml to 2.5 mg/ml in MEPC4 in an amber vial with stir bar.

MEPC4 composition=45% Cremophor RH40, 27% PEG400, 18% Corn Oil Glycerides+10% Ethanol.

Compound C was dosed p.o. as a suspension in a vehicle of 0.5% HPC and 0.2%

Tween80 in distilled water at pH8; trametinib was formulated at 0.03 and 0.0015 mg/mL.

### c) Cell Culture

The SKMEL-30 human melanoma tumor cell lines were purchased from ATCC and were included in the Novartis Cell Line Encyclopedia (CLE) cell line collection. The lines have been shown to be free of Mycoplasma sp, and murine viruses in the IMPACT-VIII PCR assay panel (IDEXX BioResearch), Columbia, MO).

The SKMEL-30 cells were maintained in RPMI 1640 (11875-093); all media were supplemented with 10% FBS (Gibco #26140-079) (56° C. for 30 min. inactivated), at 37° C. in a humidified atmosphere containing 5% carbon dioxide. Cells were harvested at 80-95% confluence, washed with PBS and detached with 0.25% trypsin-EDTA (Gibco 425200-056), neutralized with growth medium, after centrifugation for 5 min at 1200 rpm, followed by resuspension of the cell pellet in cold HBSS (Gibco #14175-095) and then mixed with an equal volume of Matrigel™ Matrix (Corning #354234) to prepare a final concentration of 50×106 cells/ml for SKMEL-30. Then 100 μl 5×10⁶ cells) was implanted subcutaneously into the right flank of the female nude mice. Tumor volume was determined by measurement with calipers and was calculated by a modified ellipsoid formula, where tumor volume (TV) (mm³)=[((1×w²)×3.14159))/6], where 1 is the longest axis of the tumor and w is perpendicular to 1. Mice were monitored for tumor growth, body weight and well-being condition twice weekly.

### Efficacy Study Design in SKMEL-30 Xenograft Model

Study to determine anti-tumor efficacy of the combination of Compound A with Compound C (trametinib) was conducted in the SKMEL-30 human melanoma model in mice as indicated in Table 1-1. Treatments were administered at a dose volume of 10 mL/kg. Mice were randomized into treatment groups on day 12 following, tumor implantation, when the average tumor volume was 190 mm³. Anti-tumor activity was determined on day 34 post tumor cell implantation; 22 days post initiation of treatment.

TABLE 1-1

| | | Compound | | | |
|---|---|---|---|---|---|
| Group | N | name | Dose (mg/kg) | Route | Schedule |
| 1 | 9 | Vehicle | 0 | p.o. | qd |
| 2 | 9 | Compound A | 25 | p.o. | bid |
| 3 | 9 | Compound C | 0.3 | p.o. | qd |
| 4 | 9 | Compound A | 25 + 0.15 | p.o. | bid + qd |
| | | plus Compound | | | |
| | | C | | | |

Vehicle = untreated; p.o. (orally): per os (oral gavage); qd (or QD): once a day; bid (or BID): twice a day.

### Data Analysis

Body Weight: The percent change in body weight was calculated as (BWcurrent−Winitial)/(BWinitial)×100%. Data was presented as mean percent body weight change from the day of treatment initiation+SEM. The percent change in body weight was calculated as (BWcurrent−BWinitial)/(BWinitial)×100%. Data was presented as mean percent body weight change from the day of treatment initiation±SEM.

Tumor Volume: Percent treatment/control (%T/C) values were calculated using the following formula:

%T/C)=100×ΔT/ΔC if ΔT≥0

%Regression=100×ΔT/Tᵢₙᵢₜᵢₐₗ if ΔT<0

Where:

T=mean tumor volume of the drug-treated group on the final day of the study;

ΔT mean tumor volume of the drug-treated group on the final day of the study—mean tumor volume of the drug-treated group on initial day of dosing;

Tᵢₙᵢₜᵢₐₗ=mean tumor volume of the drug-treated group on initial day of dosing;

C=mean tumor volume of the control group on the final day of the study; and

VC=mean tumor volume of the control group on the final day of the study mean tumor volume of the control group on initial day of dosing.

All data were expressed as mean±standard error of the mean (SEM). Between groups, comparisons were carried out using Kruskal-Wallis non-parametric ANOVA with Dunn's post-hoc test. For all statistical evaluations the level of significance was set at p<0.05.

Significance compared to the vehicle control group is reported unless otherwise stated. Survival analysis was generated by recording the day at which a mouse in each group reached a tumor burden of approximately 700 mm³. Between group comparison of survival curves were carried out using Log Rank (Mantel cox) test.

### Results

The anti-tumor efficacy of Compound A when combined with trametinib was assessed in the NRAS/BRAF double mutant (NRASQ61K/BRAFD287H) SKMEL-30 human melanoma xenograft model in athymic nude mice. Mice were treated with vehicle, single agent Compound A at 25 mg/kg orally (p.o.) twice daily (bid); single agent trametinib (Compound C) at 0.3 mg/kg orally once daily (qd), and a combination of Compound A at 25 mg/kg p.o. bid with trametinib at 0.15 mg/kg, p.o. qd. Anti-tumor activity, mean change in tumor volume, mean percent change in body weight and survival 34 days post-implantation (23 days post treatment initiation) are reported in Table 1-2. On day 34. the last day that the vehicle treated group was on study, Compound A treatment resulted in 5% tumor regression, while 0.3 mg/kg qd of trametinib resulted in 8% T/C. The combination of Compound A with trametinib at 0.15 mg/kg qd led to a further increased anti-tumor activity of 48% tumor regression when compared to the vehicle treated group (Table 1-2). 3A). The anti-tumor activity of the Compound A+trametinib combination was also significantly improved when compared to each single agent (Table 1-2). All treatment groups were well tolerated with minimal body weight loss for the duration of the study. Single agent groups were dosed continuously for the duration of the study; in the Compound A and trametinib combination group, a brief dosing holiday of trametinib only (day 28 till day 31) was provided after which the full combination was resumed to the end of the study

TABLE 1-2

| | | | | Change | | |
|---|---|---|---|---|---|---|
| | | | | in tumor | Change in | |
| | Dose | | | volume | body | |
| | (mg/kg) | | | (mm3) | weight (%) | |
| | and | | % Tumor | Mean+/− | Mean +/− | Survivor |
| Drug | schedule | T/C | Regression | SEM | SEM | (Survivors/total) |
| Vehicle | | | | 670.48 ± | 6.27 ± 1.46 | 9/9 |
| | | | | 128.12 | | |
| LXH254 | 25 bid | | 5* | −11.71 ± | 1.43 ± 1.29 | 9/9 |
| | | | | 17.01 | | |
| Trame- | 0.3 qd | 8 | | 56.95 ± | 2.45 ± 1.01 | 9/9 |
| tinib | | | | 31.05 | | |
| LXH254+ | 25 bid | | 48**# | −91.89 ± | −4.27 ± 1.03 | 9/9 |
| Trame- | +0.15 qd | | | 8.72 | | |
| tinib | | | | | | |

The studies described above demonstrated that in the NRAS/BRAF mutant melanoma xenograft SKMEL-30 in mice, antitumor efficacy was much improved by the combined treatments of Compound A with trametinib. Whilst treatment with trametinib as sole agent was accompanied by tumor growth, combined treatment with Compound A and trametinib resulted in a significant tumor regression, i.e. a 10-fold increase in the tumor regression found with treatment with Compound A as single agent. All treatments, including treatment with the combination, were tolerated as judged by lack of body weight loss for the duration of the study.

These data suggest that the combined activity of Compound A and trametinib may achieve greater and more durable responses in patients whose melanomas have MARK pathway activation.

### Example 2: Combination Efficacy of Compound A (Also Known as LXH254) and Compound C (Trametinib) in NRAS Mutant Melanoma Patient Derived Xenografts

The antitumor efficacy of Compound A when combined with trametinib was determined as followed using ten NRAS mutant patient derived melanoma xenograft models in nude mice: HMEX5727 (NRAS^{Q61K}), HMEX3486 (NRAS^{Q61K}), HMEX20667 (NRAS^{Q61R}), HMEX2921 (NRAS^{Q61K}), HMEX21684 (NRAS^{Q61K}), HMEX20585 (NRAS^{Q61R}), HMEX20864 (NRAS^{Q61R}), HMEX21124 (NRAS^{Q61H}), HMEX20744 (NRAS^{Q61K}), and HMEX4339 (NRAS^{Q61R}). Mice in eight of the models were treated for 90 days or until tumor size in each group reached >/1=700mm³. At the time of writing, mice from two models (HMEX4339, and HMEX20744) were only treated until best response was achieved, and therefore were not included in survival analysis.

### Methods

Outbred athymic (nu/nu) female mice (“HSD: Athymic Nude-nu”) (Charles River, Indianapolis) were allowed to acclimate in the Novartis NIBR animal facility with access to food and water ad libitum for minimum of 3 days prior to manipulation. Animals were handled in accordance with Novartis NIBR ACUC regulations and guidelines.

Compound A was dosed p.o. in MEPC4 vehicle (45% Cremophor RH40+27% PEG400+18% Capmul MCM C8+10% ethanol) and formulated at 5 mg/mL.

Compound C (trametinib) was dosed p.o. in a vehicle of 0.5% HPC and 0.2% Tween80 in distilled water at pH8; Trametinib was formulated at 0.03, 0.0075, and 0.000375 mg/mL.

HMEX5727, HMEX3486, HMEX20667, HMEX2921, HMEX21684, HMEX20864, HMEX20585, HMEX4339, HMEX20744, and HMEX21124 patient-derived tumor xenografts (PDX) were propagated by serial passage of tumor slurry in nude mice. Briefly, fragments of fresh tumor from a previous passage were homogenized using gentleMACS Dissociator (MACS (Miltenyi Biotec, #120-005-331), passed through a tissue grinder (Chemglass lifeSciences #CLS-5020-085), diluted in PBS, and mixed with an equal volume of Matrigel™ Matrix (Corning #354234). Then 200 ul of tumor slurry was implanted subcutaneously into the right flank of female nude mice. Tumor volume was determined by measurement with calipers and calculated using a formula, where tumor volume (Vt) (mm³)=(1×w²)/2, where 1 is the longest axis of the tumor and w is perpendicular to 1. Mice were monitored for tumor growth, body weight and body condition twice/week.

The efficacy study design for all models is described in Table 2-1. Test agents were dosed at dose volume of 10 mL/kg which was adjusted according to body weight. Tumor dimensions and body weights were collected at the time of randomization and twice weekly thereafter for the study duration. Mice were randomized into treatment groups (n=3-5/group) when the average tumor volume was approximately 350 mm³, and treatments were carried out until tumor outgrowth (tumor volume>/=700 mm³) or 90 days in 8/10 models. Percent change in tumor volume was determined for all models by comparing tumor volume change at time t to its baseline. The best response was the minimum value of percent tumor volume change for t≥10 days. At the time the untreated control mice were sacrificed, 2 mice from each group were also sacrificed, and the tumors collected for future PD analysis. The efficacy was carried out with 3 mice/group beyond this point.

TABLE 2-1
Treatment groups for efficacy studies

| | Number | | | | |
|---|---|---|---|---|---|
| Group | of mice | Compound | Dose (mg/kg) | Route | Schedule |
| 1 | 3-5 | Untreated | — | p.o. | — |
| 2 | 3-5 | Compound A | 50 | p.o. | bid |
| 3 | 3-5 | trametinib | 0.3 | p.o. | qd |
| 4 | 3-5 | Compound A and | 50 + 0.0375 or | p.o. | bid + qd |
| | | trametinib | 50 + 0.075 | | |

p.o.: per os (oral gavage)
qd: once a day
bid: twice a day

### Data Analysis

The percent change in body weight was calculated as (BW_{current}−BWᵢₙᵢₜᵢₐₗ)/(BWᵢₙᵢₜᵢₐₗ)×100%.

Data was presented as mean percent body weight change from the day of treatment initiation±SEM.

Percent change in tumor volume was determined by comparing tumor volume change at time t to its baseline using the following, formula: % tumor volume change=ΔVt=100%×((Vt−Vinitial)/Vinitial). The best response was the minimum value of ΔVt for t≥10 d.

Where:

ΔVt=change in tumor volume

Vt=tumor volume of the drug-treated (or untreated) group on a given day of the study;

Vinitial=tumor volume of the drug treated (or untreated) group on initial day of dosing. Best response >=−30% was considered (parlor regression.

Kaplan-Meier survival plot was generated using GraphPad Prism software for individual mice that reached an end point of tumor size >/=700 mm³. Statistical analysis for significance between groups was performed using Log-rank (Mantel-Cox) test, p<0.05 was considered significant.

### Results

Percent change in tumor volume (best response), percent change in body weight, and survival are reported in Table 2-2.

The combined activity of Compound A and trametinib dosed at either 50 mg/kg bid (Compound A)+0.075 mg/kg qd (trametinib) or 50 mg/kg bid (Compound A)+0.0375 mg/kg qd (trametinib) led to tumor regression in 60% of the models tested. In comparison, neither single agent Compound A dosed at 50 mg/kg bid nor single agent trametinib dosed at 0.3 mg/kg qd (i.e. at a higher dose of trametinib single agent than in the combination treatment) achieved tumor regression in any of the models tested (Table 2.2), In addition, the combination of Compound A and trametinib led to significantly increased median survival (49 days), compared to each single agents (Compound A=12 days; trametinib=7 days) or untreated controls (7 days).

Both single agents and combination treatments were generally well tolerated across models with the exception of several mice. One mouse treated with Compound A and four mice treated with the combination (spread across different studies) were sacrificed at an earlier time point due to body weight loss.

TABLE 2.2
Anti-tumor efficacy and tolerability of Compound A and trametinib in ten patient derived
NRAS mutant melanoma tumor xenograft models in mice

| | | | | | % change in | | |
|---|---|---|---|---|---|---|---|
| | | | Days | Days | tumor | % change in | |
| | | Dose | post | post | volume | body weight | |
| Model | Drug | (mg/kg) | implant | treatment | Mean +/SEM | Mean +/SEM | Survival |
| HMEX | Vehicle | | 40 | 12 | 204.23 ± | 2.50 ± 1.52 | 3/3 |
| 20585 | | | | | 70.46 | | |
| | Compound | 50 bid | 40 | 12 | 60.20 ± | 2.30 ± 1.26 | 3/3 |
| | A | | | | 34.18 | | |
| | tramctinib | 0.3 qd | 40 | 12 | 152.68 ± | 0.69 ± 2.18 | 3/3 |
| | | | | | 28.99 | | |
| | Compound | 50 bid+ | 40 | 12 | −5.79 ± | −0.07 ± 2.48 | 3/3 |
| | A | 0.075 qd | | | 13.0 | | |
| | + | | | | | | |
| | trametinib | | | | | | |
| HMEX | Vehicle | | 35 | 11 | 223.97 ± | 6.30 ± 0.48 | 5/5 |
| 20677 | | | | | 22.49 | | |
| | Compound | 50 bid | 35 | 11 | 118.78 ± | 2.04 ± 1.48 | 5/5 |
| | A | | | | 23.65 | | |
| | trametinib | 0.3 qd | 35 | 11 | 140.14 ± | 0.42 ± 1.36 | 5/5 |
| | | | | | 21.43 | | |
| | Compound | 50 bid+ | 55 | 31 | −76.03 ± | −2.46 ± 2.71 | 3/3 |
| | A | 0.075 qd | | | 2.30 | | |
| | +trametinib | | | | | | |
| HMEX | Vehicle | | 20 | 7% | 372.45 ± | −1.64 ± 0.80 | 5/5 |
| 5727 | | | | | 79.63 | | |
| | Compound | 50 bid | 20 | 7* | 161.18 ± | −5.11 ± 1.35 | 5/5 |
| | A | | | | 34.37 | | |
| | trametinib | 0.3 qd | 20 | 7* | 323.88 ± | −6.42 ± 2.50 | 5/5 |
| | | | | | 71.7 | | |
| | Compound | 50 bid+ | 44 | 31 | −93.16 ± | 7.77 ± 11.91 | 2/3 |
| | A | 0.075 qd | | | 6.84 | | |
| | +trametinib | | | | | | |
| HMEX | Vehicle | | 45 | 12 | 303.57 ± | 3.13 ± 0.84 | 3/3 |
| 2921 | | | | | 78.08 | | |
| | Compound | 50 bid | 45 | 12 | 148.85 ± | −6.79 ± 1.48 | 3/3 |
| | A | | | | 16.77 | | |
| | trametinib | 0.3 qd | 45 | 12 | 205.31 ± | −2.30 ± 0.82 | 3/3 |
| | | | | | 11.58 | | |
| | Compound | 50 bid+ | 45 | 12 | 11.36 ± | 1.20 ± 0.34 | 3/3 |
| | A | 0.075 qd | | | 2.96 | | |
| | +trametinib | | | | | | |
| HMEX | Vehicle | | 22 | 8* | 358.71 ± | 1.60 ± 1.75 | 5/5 |
| 3486 | | | | | 31.62 | | |
| | Compound | 50 bid | 26 | 12 | 146.22 ± | 2.52 ± 1.14 | 5/5 |
| | A | | | | 27.86 | | |
| | trametinib | 0.3 qd | 26 | 12 | 317.02 ± | −0.26 ± 2.67 | 5/5 |
| | | | | | 92.42 | | |
| | Compound | 50 bid+ | 40 | 26 | 2.54 ± | 7.26 ± 1.26 | 3/3 |
| | A | 0.075 qd | | | 17.88 | | |
| | +trametinib | | | | | | |
| HMEX | Vehicle | | 28 | 9* | 321.61 ± | 3.84 ± 0.93 | 5/5 |
| 21684 | | | | | 99.82 | | |
| | Compound | 50 bid | 33 | 14 | 314.39 ± | 2.64 ± 2.43 | 3/3 |
| | A | | | | 111,8 | | |
| | trametinib | 0.3 qd | 33 | 14 | 229.68 ± | −2.31 ± 2.24 | 3/3 |
| | | | | | 30.38 | | |
| | Compound | 50 bid+ | 33 | 14 | 40.51 ± | −3.67 ± 2.02 | 3/3 |
| | A | 0.075 qd | | | 25.93 | | |
| | +trametinib | | | | | | |
| HMEX | Vehicle | | 30 | 11 | 278.81 ± | −1.26 ± 1.53 | 5/5 |
| 20864 | | | | | 34.23 | | |
| | Compound | 50 bid | 30 | 11 | 96.72 ± | −0.42 ± 2.28 | 5/5 |
| | A | | | | 27.35 | | |
| | trametinib | 0.3 qd | 30 | 11 | 211.65 ± | −3.77 ± 1.80 | 5/5 |
| | | | | | 24.30 | | |
| | Compound | 50 bid+ | 47 | 28 | −32.29 ± | −3.59 ± 1.13 | 3/3 |
| | A | 0.0375 | | | 17.69 | | |
| | +trametinib | qd | | | | | |
| HMEX | Vehicle | | 55 | 10 | 112.05 ± | −0.30 ± 1.27 | 5/5 |
| 21124 | | | | | 31.69 | | |
| | Compound | 50 bid | 55 | 10 | 31.99 ± | −3.36 ± 0.75 | 5/5 |
| | A | | | | 32.74 | | |
| | tramctinib | 0.3 qd | 55 | 10 | 76.31 ± | −3.22 ± 1.05 | 5/5 |
| | | | | | 21.75 | | |
| | Compound | 50 bid+ | 55 | 10 | −47.59 ± | −1.75 ± 1.35 | 5/5 |
| | A | 0.0375 | | | 7.64 | | |
| | +trametinib | qd | | | | | |
| HMEX | Vehicle | | 70 | 11 | 159.94 ± | 2.74 ± 1.52 | 3/3 |
| 20744 | | | | | 38.27 | | |
| | Compound | 50 bid | 83 | 24 | −6.14 ± | 6.14 ± 3.50 | 3/3 |
| | A | | | | 24.14 | | |
| | trametinib | 0.3 qd | 70 | 11 | 70.56 ± | 0.54 ± 1.64 | 3/3 |
| | | | | | 9.40 | | |
| | Vehicle# | | 55 | 10 | 186.05 ± | 5.67 ± 0.68 | 3/3 |
| | | | | | 40.04 | | |
| | Compound | 50 bid+ | 70 | 25 | −87.62 ± | 0.57 ± 4.04 | 3/3 |
| | A# | 0.0375 qd | | | 2.74 | | |
| | +trametinib | | | | | | |
| HMEX | Vehicle | | 48 | 10 | 93.41 ± | −3.66 ± 0.86 | 3/3 |
| 4339 | | | | | 7.92 | | |
| | Compound | 50 bid | 48 | 10 | 9.35 ± | 6.28 ± 4.04 | 3/3 |
| | A | | | | 16.00 | | |
| | trametinib | 0.3 qd | 48 | 10 | 19.17 ± | 5.87 ± 2.04 | 3/3 |
| | | | | | 25.39 | | |
| | Compound | 50 bid+ | 70 | 32 | −55.86 ± | −2.32 ± 4.77 | 3/3 |
| | A | 0.0375 | | | 6.70 | | |
| | +trametinib | qd | | | | | |

*indicates last day of treatment (tumor size >/= 700 mm³)
#group run in a separate experiment

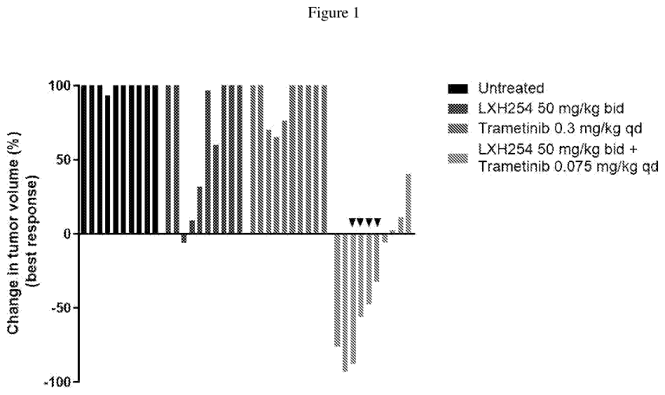

A waterfall plot of responses to Compound A (LXH254) and trametinib across patient derived NRAS^{mut} melanoma tumor xenograft models in mice is shown in FIG. 1. In FIG. 1, each bar represents the best response achieved by each treatment (plotted as the average of 3-5 mice/treatment) in an individual PDX. Arrowheads indicate models that were treated with Compound A 50 mg/kg bid+trametinib 0.0375 mg/kg qd. Models for each treatment are plotted left to right in the following order: HMEX20667, HMEX5727, HMEX20744, HMEX4339, HMEX21124, HMEX20864, HMEX20585, HMEX3486, HMEX2920, and HMEX21684.

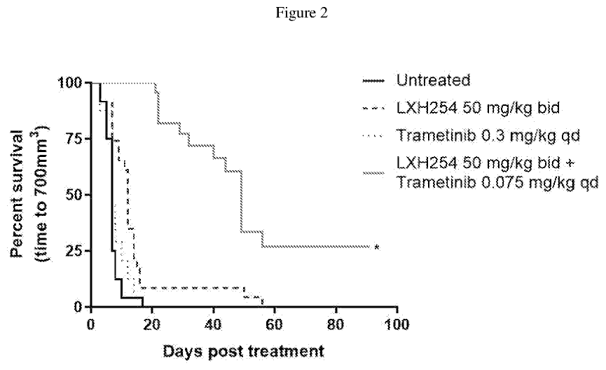

FIG. 2 is Kaplan-Meier plot of time that tumors reached a size of 700 mm³ during daily treatment of single agents Compound A, trametinib or combination of both. Treatment was initiated when average tumor size of each model was around 350 mm³, and all animals received continuous daily drug treatments for the duration of the study. Study was terminated when animals were treated for 90 days or reached a tumor size of >/=700 mm³. Survival curves of the combination group was statistically significant [*p<0.05 Log-rank (Mantel-Cox test)] when compared to each single agent or untreated controls,

The combined activity of Compound A and trametinib dosed at either 50 mg/kg bid (Compound A)+mg/kg qd (trametinib) or 50 mg/kg bid (Compound A )+0.0375 mg/kg qd (trametinib) led to tumor regression in 60% of the models tested. In comparison, neither single agent Compound A dosed at 50 mg/kg bid nor single agent trametinib dosed at 0.3 mg/kg qd achieved tumor regression in any of the models tested. In addition, the combination of Compound A and trametinib led to significantly increased median percent survival, compared to each single agent or untreated controls. Collectively, these data indicate that the combination of Compound A and trametinib may achieve greater and more durable responses in NRAS mutant melanoma patients.

### Example 3: Combination Efficacy of Compound A (Also Known as LXH254) and Compound B in NRAS mutant Melanoma Patient Derived Xenografts

The antitumor efficacy of Compound A when combined with Compound B was determined as followed using NRAS mutant patient derived melanoma xenograft models in nude mice. The antitumor efficacy of Compound A when combined with Compound B was determined using ten NRAS mutant patient derived melanoma xenograft models in nude mice: HMEX5727 (NRAS^{Q61K}), HMEX3486 (NRAS^{Q61K}), HMEX20667 (NRAS^{Q61R}), HMEX2921 (NRAS^{Q61K}), HMEX21684 (NRAS^{Q61K}), HMEX20585 (NRAS^{Q61R}), HMEX20864 (NRAS^{Q61R}), HMEX21086 (NRAS^{Q61H}), HMEX20744 (NRAS^{Q61K}), and HMEX4339 (NRAS^{Q61R}). Mice were treated for approximately 90-100 days or until tumor size in each group reached >/=700 mm³.

### Methods

Outbred athymic (nu/nu) female mice (“HSD: Athymic Nude-nu”) (Charles River, Indianapolis) were allowed to acclimate in the Novartis NIBR animal facility with access to food and water ad libitum for minimum of 3 days prior to manipulation. Animals were handled in accordance with Novartis NIBR ACUC regulations and guidelines.

Compound A was dosed p.o. in MEPC4 vehicle (45% Cremophor RH40+27% PEG400+18% Corn Oil Glycerides (Maisine CC)+10% ethanol). MEPC 4 stocks was diluted 5×(1:4 with DI Water) prior to dosing. Compound A was formulated at 2.5 mg/mL and 5 mg/mL.

Compound B was dosed p.o. in a vehicle of 0.5% HPC+0.5% Pluronic F-127 in Phosphate Buffer pH 5. Final pH is adjusted to 4-4.5. Compound B was formulated at 1.5 mg/mL and 7.5 mg/mL All patient-derived tumor xenografts (PDX) were propagated by serial passage of tumor slurry in nude mice as described in Example 2. Tumor volume was determined as in Example 2. Mice were monitored for tumor growth, body weight and body condition twice/week.

Test agents were dosed at dose volume of 10 mL/kg which was adjusted according to body weight. Tumor dimensions and body weights were collected at the time of randomization and twice weekly thereafter for the study duration. Mice were randomized into treatment groups (n=3-5/group) when the average tumor volume was approximately 350 mm³, and treatments were carried out until tumor outgrowth (approximate tumor volume 700 mm³) in 9 models. Treatment for model HMEX5727 was discontinued after 21 days and data from this model was not included in the Kaplan-Meier analysis. Percent change in tumor volume was determined for all models by comparing tumor volume change at time t to its baseline. The best response was the minimum value of percent tumor volume change at t≥10 days. At the time the untreated control mice were sacrificed, 2 mice from each group were also sacrificed, and the tumors collected for future pharmacodynamic analysis. The efficacy was carried out with 2-3 mice/group beyond this point.

TABLE 3-1
Treatment groups for efficacy studies

| | Number | | | | |
|---|---|---|---|---|---|
| Group | of mice | Compound | Dose (mg/kg) | Route | Schedule |
| 1 | 3-5 | Untreated | — | p.o. | — |
| 2 | 3-5 | Compound A | 50 | p.o. | bid |
| 3 | 3-5 | Compound B | 75 | p.o. | qd |
| 4 | 3-5 | Compound A and | 50 + 15 or | p.o. | bid + qd |
| | | Compound B | 25 + 75 | | |

p.o.: per os (oral gavage)
qd: once a day
bid: twice a day

### Data Analysis

Data analysis was carried out as described in Example 2.

Kaplan-Meier survival plot was generated using GraphPad Prism software for individual mice that reached an end point of approximate tumor size 700 mm3. Statistical analysis for significance between groups was performed using Log-rank (Mantel-Cox) test. p<0.05 was considered significant.

### Results

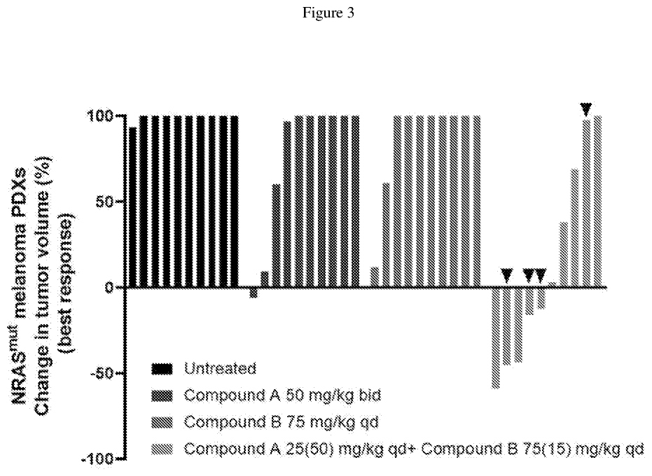

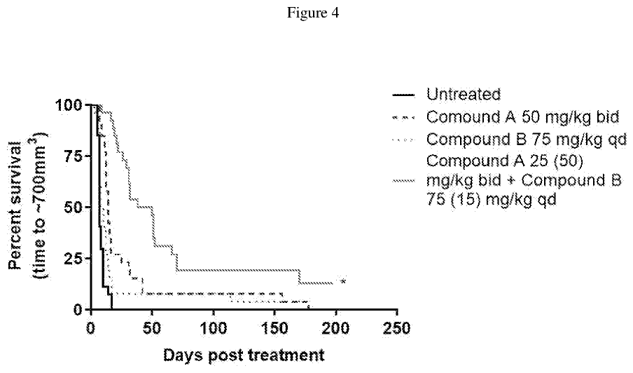

Percent change in tumor volume (best response), percent change in body weight, and survival are reported in Table 3-2, FIG. 3 and FIG. 4.

The combined activity of Compound A and Compound B dosed at either 50 mg/kg bid (Compound A)+15 mg/kg qd (Compound B) or 25 mg/kg bid (Compound A)+75 mg/kg qd (Compound B) led to tumor regression in 30% of the models tested. It is to be noted that even though in the combination treatments, Compound A or Compound B was dosed at a either half or a third of the amounts used in the single agent experiments, neither single agent Compound A dosed at 50 mg/kg bid nor single agent Compound B dosed at 75 mg/kg qd achieved tumor regression in the models tested (Table 3-2 and FIG. 3).

In addition, the combination of Compound A and Compound B led to significantly increased median survival (44 days), compared to each single agents (Compound. A=14 days; Compound B=9 days) or untreated controls (7 days) (FIG. 4).

Both single agents and combination treatments were well tolerated across models.

TABLE 3-2
Anti-tumor efficacy and tolerability of Compound A and Compound B in ten patient
derived NRAS mutant melanoma tumor xenograft models in mice

| | | | Days | | % change in | % change in | |
|---|---|---|---|---|---|---|---|
| | | Dose | post | Days post | tumor volume | body weight | |
| Model | Drug | (mg/kg) | implant | treatment | Mean +/SEM | Mean +/SEM | Survival |
| HMEX | Untreated | | 40 | 12 | 204.23 ± | 2.50 ± 1.52 | 3/3 |
| 20585 | | | | | 70.46 | | |
| | Compound | 50 bid | 40 | 12 | 60.20 ± | 2.30 ± 1.26 | 3/3 |
| | A | | | | 34.18 | | |
| | Compound | 75 qd | 40 | 12 | 207.58 ± | 1.39 ± 1.20 | 3/3 |
| | B | | | | 29.36 | | |
| | Compound | 25 bid+ | 40 | 12 | 97.62 ± | −1.65± 0.38 | 3/3 |
| | A + | 75 qd | | | 48.68 | | |
| | Compound | | | | | | |
| | B | | | | | | |
| HMEX | Untreated | | 35 | 11 | 223.97 ± | 6.30 ± 0.48 | 5/5 |
| 20677 | | | | | 22.49 | | |
| | Compound | 50 bid | 35 | 11 | 118.78 ± | 2.04 ± 1.48 | 5/5 |
| | A | | | | 23.65 | | |
| | Compound | 75 qd | 35 | 11 | 171.57 ± | 4.32 ± 0.30 | 5/5 |
| | B | | | | 9.25 | | |
| | Compound | 25 bid+ | 35 | 11 | −12.36 ± | −5.14 ± 0.89 | 5/5 |
| | A + | 75 qd | | | 12.25 | | |
| | Compound | | | | | | |
| | B | | | | | | |
| HMEX | Untreated | | 20 | 7* | 372.45 ± | −1.64 ± 0.80 | 5/5 |
| 5727 | | | | | 79.63 | | |
| | Compound | 50 bid | 20 | 7* | 161.18 ± | −5.11 ± 1.35 | 5/5 |
| | A | | | | 34.37 | | |
| | Compound | 75 qd | 20 | 7* | 246.70 ± | −2.37 ± 1.79 | 4/4 |
| | B | | | | 34.37 | | |
| | Compound | 25 bid+ | 31 | 18 | −16.20 ± | 5.15 ± 8.75 | 2/4 |
| | A + | 75 qd | | | 33.24 | | |
| | Compound | | | | | | |
| | B | | | | | | |
| HMEX | Untreated | | 45 | 12 | 303.57 ± | 3.13 ± 0.84 | 3/3 |
| 2921 | | | | | 78.08 | | |
| | Compound | 50 bid | 45 | 12 | 148.85 ± | −6.79 ± 1.48 | 3/3 |
| | A | | | | 16.77 | | |
| | Compound | 75 qd | 45 | 12 | 196.00 ± | 5.16 ± 0.96 | 3/3 |
| | B | | | | 67.19 | | |
| | Compound | 50 bid+ | 45 | 12 | 38.11 ± | 6.32 ± 1.80 | 3/3 |
| | A + | 15 qd | | | 10.91 | | |
| | Compound | | | | | | |
| | B | | | | | | |
| HMEX | Untreated | | 22 | 8* | 358.71 ± | 1.60 ± 1,75 | 5/5 |
| 3486 | | | | | 31.62 | | |
| | Compound | 50 bid | 26 | 12 | 146.22 ± | 2.52 ± 1.14 | 5/5 |
| | A | | | | 27.86 | | |
| | Compound | 75 qd | 26 | 12 | 170.26 ± | 3.90 ± 1.38 | 5/5 |
| | B | | | | 51.28 | | |
| | Compound | 25 bid+ | 43 | 29 | −45.34 ± | 5.48 ± 1.21 | 3/5 |
| | A + | 75 qd | | | 6.91 | | |
| | Compound | | | | | | |
| | B | | | | | | |
| HMEX | Untreated | | 28 | 9* | 321.61 ± | 3.84 ± 0.93 | 5/5 |
| 21684 | | | | | 99.82 | | |
| | Compound | 50 bid | 33 | 14 | 314.39 ± | 2.64 ± 2.43 | 3/5 |
| | A | | | | 111.8 | | |
| | Compound | 75 qd | 33 | 14 | 247.55 ± | −2.15 ± 2.90 | 3/5 |
| | B | | | | 66.41 | | |
| | Compound | 50 bid+ | 33 | 14 | 111.83 ± | 1.51 ± 1.21 | 3/5 |
| | A + | 15 qd | | | 58.63 | | |
| | Compound | | | | | | |
| | B | | | | | | |
| HMEX | Untreated | | 30 | 11 | 278.81 ± | −0.07 ± 1.24 | 4/4 |
| 20864 | | | | | 34.23 | | |
| | Compound | 50 bid | 30 | 11 | 96.72 ± | −0.42 ± 2.28 | 5/5 |
| | A | | | | 27.35 | | |
| | Compound | 75 qd | 30 | 11 | 205.98 ± | −3.52 ± 1.31 | 5/5 |
| | B | | | | 30.28 | | |
| | Compound | 50 bid+ | 61 | 42 | −58.85 ± | −0.79 ± 2.51 | 3/5 |
| | A + | 15 qd | | | 7.02 | | |
| | Compound | | | | | | |
| | B | | | | | | |
| HMEX | Untreated | | 55 | 10 | 168.56 ± | 4.81 ± 1.31 | 5/5 |
| 21086 | | | | | 17.66 | | |
| | Compound | 50 bid | 55 | 10 | 58.65 ± | 3.72 ± 0.77 | 5/5 |
| | A | | | | 15.39 | | |
| | Compound | 75 gd | 55 | 10 | 152.01 ± | 4.64 ± 1.67 | 5/5 |
| | B | | | | 11.66 | | |
| | Compound | 50 bid+ | 55 | 10 | 68.89 ± | 3.07 ± 1.55 | 5/5 |
| | A + | 15 qd | | | 10.70 | | |
| | Compound | | | | | | |
| | B | | | | | | |
| HMEX | Untreated | | 70 | 11 | 159.94 ± | 2.74 ± 1.52 | 3/3 |
| 20744 | | | | | 38.27 | | |
| | Compound | 50 bid | 83 | 24 | −6.14 ± | 6.14 ± 3.50 | 3/3 |
| | A | | | | 24.14 | | |
| | Compound | 75 qd | 87 | 24 | 11.65 ± | 3.40 ± 1.73 | 3/3 |
| | B | | | | 28.95 | | |
| | Untreated # | | 55 | 10 | 186.06 ± | 5.67 ± 0.68 | 3/3 |
| | | | | | 40.04 | | |
| | Compound | 50 bid+ | 62 | 17 | −43.68 ± | 5.13 ± 4.47 | 3/5 |
| | A# + | 15 qd | | | 6.58 | | |
| | Compound | | | | | | |
| | B | | | | | | |
| HMEX | Untreated | | 48 | 10 | 93.41 ± | −3.66 ± 0.86 | 3/3 |
| 4339 | | | | | 7.92 | | |
| | Compound | 50 bid | 48 | 10 | 9.35 ± | 6.28 ± 4.04 | 3/3 |
| | A | | | | 16.00 | | |
| | Compound | 75 qd | 48 | 10 | 60.66 ± | 2.33 ± 0.57 | 3/3 |
| | B | | | | 24.93 | | |
| | Compound | 50 bid+ | 48 | 10 | 3.36 ± | 4.34 ± 1.77 | 5/5 |
| | A + | 15 qd | | | 16.00 | | |
| | Compound | | | | | | |
| | B | | | | | | |

*indicates last day of treatment (tumor size >/= 700 mm³)
#group run in a separate experiment

FIG. 3 is a waterfall plot of responses to Compound A and Compound B across ten patient derived NRASmut melanoma tumor xenograft models in mice. Each bar represents the best response achieved by each treatment (plotted as the average of 2-5 mice/treatment) in an individual patient derived xenograft (PDX). Arrowheads indicate models that were treated with Compound A 25 mg/kg bid+Compound B 75 mg/kg qd. FIG. 4 is a Kaplan-Meier plot of time that tumors reached a size of ˜700 mm³ during daily treatment of single agents Compound A, Compound B or combination of both. Treatment was initiated when average tumor size of each model was around 350mm ³, and all animals received continuous daily drug treatments for the duration of the study. Study was terminated when animals reached an approximate tumor size of ˜700 mm³. Survival curves of the combination group was statistically significant [*p=0.05 Log-rank (Mantel-Cox test)] when compared to each single agent or untreated controls.

The combined activity of Compound A and Compound B dosed at either 50 mg/kg bid (Compound A)+15 mg/kg qd (Compound B) or 25 mg/kg bid (Compound A)+75 mg/kg qd (Compound B) led to tumor regression in 30% of the models tested. In comparison, neither single agent Compound A dosed at 50 mg/kg bid nor single agent Compound B dosed at 75 mg/kg qd achieved tumor regression in the models tested. In addition, the combination of Compound A and Compound B led to significantly increased median percent survival, compared to each single agent or untreated controls. Collectively, these data indicate that the combination of Compound A and Compound B may achieve greater and more durable responses in NRAS mutant melanoma patients.

Example 4: Combination Efficacy of Compound A (also known as LXH254) and Ribociclib (Compound D or LEE011)in NRAS Mutant Melanoma Patient Derived Xenografts

The antitumor efficacy of Compound A when combined with Compound C was determined as followed using NRAS mutant patient derived melanoma xenograft models in nude mice. The antitumor efficacy of Compound A when combined with Compound B was determined using nine NRAS mutant patient derived melanoma xenograft models in nude mice: HMEX5727 (NRAS^{Q61K}), HMEX3486 (NRAS^{Q61K}), HMEX20667 (NRAS^{Q61R}), HMEX2921 (NRAS^{Q61K}), HMEX21684 (NRAS^{Q61K}), HMEX20585 (NRAS^{Q61R}), HMEX20864 (NRAS^{Q61R}), HMEX21086 (NRAS^{Q61H}), HMEX20744 (NRAS^{Q61K}), and HMEX4339 (NRAS^{Q61R}). Mice were treated for approximately 90-100 days or until tumor size in each group reached >/=700mm³.

### Methods

Outbred athymic (nu/nu) female mice (“HSD: Athymic Nude-nu”) (Charles River, Indianapolis) were allowed to acclimate in the Novartis NIBR animal facility with access to food and water ad libitum for minimum of 3 days prior to manipulation. Animals were handled in accordance with Novartis NIBR ACUC regulations and guidelines.

Compound A was dosed p.o. in MEPC4 vehicle (45% Cremophor RH40+2.7% PEG400+18% Corn Oil Glycerides+10% ethanol). Compound A was formulated at 5 mg/mL.

Ribociclib was dosed p.o, in a vehicle of 0,5% methyl cellulose; ribociclib was formulated at 7.5 mg/mL. All patient-derived tumor xenografts (PDX) were propagated by serial passage of tumor slurry in nude mice as described in Example 2. Tumor volume was determined as in Example 2. Mice were monitored for tumor growth, body weight and body condition twice/week.

Test agents were dosed at dose volume of 10 mL/kg which was adjusted according to body weight. Tumor dimensions and body weights were collected at the time of randomization and twice weekly thereafter for the study duration. Mice were randomized into treatment groups (n=3-5/group) when the average tumor volume was approximately 350 mm³, and treatments were carried out until tumor outgrowth (tumor volume >/=700 mm3) or approximately 90 days. Percent change in tumor volume was determined for all models by comparing tumor volume change at time t to its baseline. The best response was the minimum value of percent tumor volume change for t≥10 days. At the time the untreated control mice were sacrificed, 2 mice from each group were also sacrificed, and the tumors collected for future PD analysis. The efficacy was carried out with 3 mice/group beyond this point.

TABLE 4-1
Treatment groups for efficacy studies

| | Number | | | | |
|---|---|---|---|---|---|
| Group | of mice | Compound | Dose (mg/kg) | Route | Schedule |
| 1 | 3-5 | Untreated | — | p.o. | — |
| 2 | 3-5 | Compound A | 50 | p.o. | bid |
| 3 | 3-5 | ribociclib | 75 | p.o. | qd |
| 4 | 3-5 | Compound A and | 50 + 75 | p.o. | bid + qd |
| 5 | | ribociclib | | | |

p.o.: per os (oral gavage)
qd: once a day
bid: twice a day

Data analysis was carried out as described in Example 2.

Kaplan-Meier survival plot was generated using GraphPad Prism software for individual mice that reached an end point of tumor size >/=700 mm³. Statistical analysis for significance between groups was performed using Log-rank (Mantel-Cox) test. p<0.05 was considered significant.

### Results

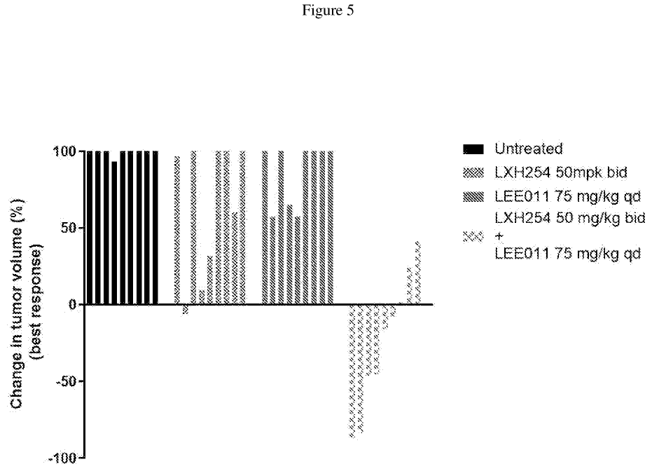

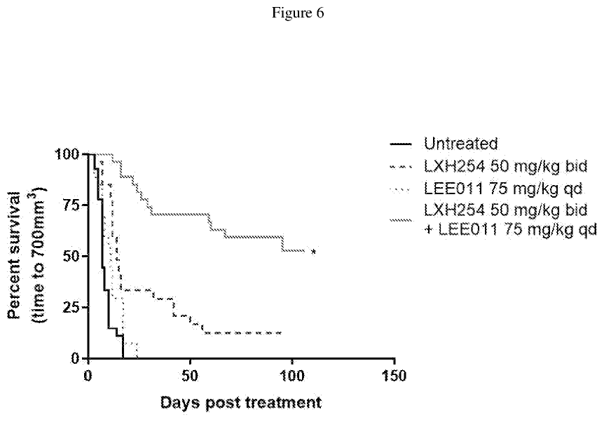

Percent change in tumor volume (best response), percent change in body weight, and survival are reported in Table 4-2 and FIG. 5 and FIG. 6.

The combined activity of Compound A and ribociclib dosed at 50 mg/kg bid (Compound A)+75 mg/kg qd (ribociclib) led to tumor regression in 44% of the models tested. In comparison, neither single agent Compound A dosed at 50 mg/kg bid nor single agent ribociclib dosed at 75 mg/kg qd achieved tumor regression in any of the models tested (Table 3-2 and FIG. 5). In addition, the combination of Compound A and ribociclib led to significantly increased median survival, compared to each single agents or untreated controls (FIG. 6).

Both single agents and combination treatments were very well tolerated as judged by lack of body weight loss across models. One mouse treated with Compound A was sacrificed at an earlier time point due to body weight loss.

TABLE 4-2
Anti-tumor efficacy and tolerability of Compound A and ribociclib in nine patient derived
NRAS mutant melanoma tumor xenograft models in mice

| | | | Days | | % change in | % change in | |
|---|---|---|---|---|---|---|---|
| | | Dose | post | Days post | tumor volume | body weight | |
| Model | Drug | (mg/kg) | implant | treatment | Mean+/SEM | Mean+/SEM | Survival |
| HMEX | Vehicle | | 40 | 12 | 204.23 ± 70.46 | 2.50 ± 1.52 | 3/3 |
| 20585 | Compound | 50 bid | 40 | 12 | 60.20 ± 34.18 | 2.30 ± 1.26 | 3/3 |
| | A | | | | | | |
| | ribociclib | 75 qd | 40 | 12 | 120.55 ± 4.43 | 1.99 ± 1.74 | 3/3 |
| | Compound | 50 bid+ | 40 | 12 | 23.93 ± 17.42 | −1.02 ± 1.50 | 3/3 |
| | A + | 75 qd | | | | | |
| | ribociclib | | | | | | |
| HMEX | Vehicle | | 35 | 11 | 223.97 ± 22.49 | 6.30 ± 0.48 | 5/5 |
| 20677 | Compound | 50 bid | 35 | 11 | 118.78 ± 23.65 | 2.04 ± 1.48 | 5/5 |
| | A | | | | | | |
| | ribociclib | 75 qd | 35 | 11 | 142.57 ± 15.02 | 2.27 ± 0,99 | 5/5 |
| | Compound | 50 bid+ | 38 | 14 | 5.42 ± 15.11 | 3.33 ± 1.74 | 5/5 |
| | A + | 75 qd | | | | | |
| | ribociclib | | | | | | |
| HMEX | Vehicle | | 20 | 7* | 372.45 ± 79.63 | −1.64 ± 0.80 | 5/5 |
| 5727 | Compound | 50 bid | 20 | 7* | 161.18 ± 34.37 | −5.11 ± 1.35 | 5/5 |
| | A | | | | | | |
| | ribociclib | 75 qd | 20 | 7* | 350.51 ± 67.22 | −6.01 ± 1.55 | 5/5 |
| | Compound | 50 bid+ | 48 | 35 | −46.41 ± 25.25 | 4.68 ± 1.10 | 3/3 |
| | A + | 75 qd | | | | | |
| | ribociclib | | | | | | |
| HMEX | Vehicle | | 45 | 12 | 303.57 ± 78.08 | 3.13 ± 0.84 | 3/3 |
| 2921 | Compound | 50 bid | 45 | 12 | 148.85 ± 16.77 | −6.79 ± 1.48 | 3/3 |
| | A | | | | | | |
| | ribociclib | 75 qd | 45 | 12 | 240.32 ± 72.55 | 4.21 ± 1.71 | 3/3 |
| | Compound | 50 bid+ | 45 | 12 | 41.35 ± 13.22 | 2.84 ± 2.57 | 3/3 |
| | A + | 75 qd | | | | | |
| | ribociclib | | | | | | |
| HMEX | Vehicle | | 22 | 8* | 358.71 ± 99.83 | 1.60 ± 1.75 | 5/5 |
| 3486 | Compound | 50 bid | 26 | 12 | 146.22 ± 27.86 | 2.52 ± 1.14 | 5/5 |
| | A | | | | | | |
| | ribociclib | 75 gd | 26 | 12 | 287.09 ± 52.45 | −1.07 ± 1.10 | 5/5 |
| | Compound | 50 bid++ | 40 | 26 | −7.82 ± 25.94 | 7.45 ± 1.27 | 3/3 |
| | A + | 75 qd | | | | | |
| | ribociclib | | | | | | |
| HMEX | Vehicle | | 30 | 11 | 278.81 ± 34.23 | −0.07 ± 1.23 | 5/5 |
| 20864 | Compound | 50 bid | 30 | 11 | 96.72 ± 27.35 | −0.42 ± 2.28 | 5/5 |
| | A | | | | | | |
| | ribociclib | 75 qd | 30 | 11 | 216.16 ± 46.98 | −0.25 ± 2.16 | 5/5 |
| | Compound | 50 bid+ | 68 | 49 | −87.38 ± 0.71 | 8.02 ± 2.17 | 3/3 |
| | A + | 75 qd | | | | | |
| | ribociclib | | | | | | |
| HMEX | Vehicle | | 55 | 10 | 112.05 ± 31.69 | −0.30 ± 1.27 | 5/5 |
| 21124 | Compound | 50 bid | 55 | 10 | 31.99 ± 32.74 | −3.36 ± 0.75 | 5/5 |
| | A | | | | | | |
| | ribociclib | 75 qd | 55 | 10 | 57.84 ± 19.83 | 2.85 ± 1.26 | 5/5 |
| | Compound | 50 bid+ | 55 | 10 | −15.84 ± 7.64 | −0.10 ± 2.00 | 5/5 |
| | A + | 75 qd | | | | | |
| | ribociclib | | | | | | |
| HMEX | Vehicle | | 70 | 11 | 159.94 ± 26.2 | 2.74 ± 1.52 | 3/3 |
| 20744 | Compound | 50 bid | 83 | 24 | −6.14 ± 24.14 | 6.14 ± 3.50 | 3/3 |
| | A | | | | | | |
| | ribociclib | 75 qd | 70 | 11 | 57.50 ± 13.94 | 4.84 ± 1.58 | 3/3 |
| | Vehicle# | | 55 | 10 | 186.05 ± 40.04 | 5.67 ± 0.68 | 3/3 |
| | Compound | 50 bid+ | 83 | 38 | −83.90 ± 3.10 | 10.62 ± 0.07 | 3/3 |
| | A# + | 75qd | | | | | |
| | ribociclib | | | | | | |
| HMEX | Vehicle | | 48 | 10 | 93.41 ± 7.92 | −3.66 ± 0.86 | 3/3 |
| 4339 | Compound | 50 bid | 48 | 10 | 9.35 ± 16.00 | 6.28 ± 4.04 | 3/3 |
| | A | | | | | | |
| | ribociclib | 75 qd | 48 | 10 | 65.18 ± 9.67 | 5.96 ± 1.97 | 3/3 |
| | Compound | 50 bid+ | 91 | 53 | −50.90 ± 8.57 | 10.99 ± 2.14 | 3/3 |
| | A + | 75 qd | | | | | |
| | ribociclib | | | | | | |

*indicates last day of treatment (tumor size >/= 700 mm³)
#group run in a separate experiment

FIG. 5 shows the anti-tumor or activity of Compound A (LXH254) and ribociclib (LEE011) across nine patient derived NRAS^{mut} melanoma tumor xenograft models in mice. Each bar represents the best response achieved by each treatment (plotted as the average of 3-5 mice/treatment) in an individual PDX. Models for each treatment are plotted left to right in the following order: HMEX20864, HMEX20744, HMEX4339, HMEX5727, HMEX21124, HMEX3486, HMEX20667, HMEX20585, and HMEX2921.

FIG. 6 is a Kaplan-Meier plot of time that tumors reached a size of 700 mm³ during daily treatment of single agents Compound A, ribociclib or combination of both. Treatment was initiated when average tumor size of each model was around 350mm³, and all animals received continuous daily drug treatments for the duration of the study. Study was terminated when animals were treated for >/=90 days or reached a tumor size of >/−700 m³. Survival curves of the combination group was statistically significant [*p<0.05 Log-rank (Mantel-Cox test)] when compared to each single agent or untreated controls.

The in vivo activity of the Compound A and ribociclib combination in NRAS mutant melanoma was profiled in a panel of nine NRAS mutant patient derived melanoma xenografts. The combined activity of Compound A and ribociclib dosed at 50 mg/kg bid (Compound A)+75 mg/kg qd (ribociclib) led to tumor regression in 44% of the models tested. In comparison, neither single agent Compound A dosed at 50 mg/kg bid nor single agent ribociclib dosed at 75 mg/kg qd achieved tumor regression in any of the models tested. In addition, the combination of Compound A and ribociclib was well tolerated and led to significantly increased median percent survival, compared to each single agent or untreated controls. Collectively, these data indicate that the combination of Compound A and ribociclib may achieve greater and more durable responses in NRAS mutant melanoma patients.

### Example 5: Study of Efficacy and Safety of Compound A in Combination With a Second Therapeutic Agent Selected From the Group Consisting of (i) Compound B, (ii) Compound C (Trametinib) and (iii) Compound D (Ribociclib) in Patients With Previously Treated Unresectable or Metastatic Melanoma

The combination of the invention may be tested as follows.

A randomized, open-label, multi-arm, two-part, phase II study to assess efficacy and safety of multiple Compound A combinations in patients with previously treated unresectable or metastatic BRAFV600 or NRAS mutant melanoma is conducted.

Each dual combination tested is expected to help overcome intrinsic and acquired resistance to BRAF targeted therapy as well as offer new treatment options for NRAS-mutant melanoma patients. This study consists of two parts:

### Part 1: Selection Part

Based on molecular testing (local or central), participants are allocated to one of two groups: BRAFV600 mutant or NRAS mutant.

Group 1: Participants with BRAFV600 mutant unresectable or metastatic melanoma

Group 2: Participants with NRAS mutant unresectable or metastatic melanoma

After the completion of screening procedures, eligible participants may be randomized within the combination aims of each respective group using an Interactive Response Technology (IRT) system. For each patient population, randomization may be stratified by baseline lactate dehydrogenase (LDH) level: LDH≤ULN (upper limit of normal), and LDH>ULN.

For example, normal LDH levels may range from 140 units per liter (U/L) to 280 U/L or 2.34 mkat/L to 4.68 mkat/L.

In Part 1, participants may be randomized with equal probability into three initial combination anus in two groups based on mutation status to either BRAFV600 or NRAS melanoma:
Group 1: Participants with BRAFV600 mutant unresectable or metastatic melanoma:
   Arm 1: Compound A 400 mg BID (twice a day) and Compound B 200 mg QD (once a day) or Compound A 200 mg BID (twice a day) and Compound B 200 mg QD (once a day)
   Arm 2: Compound A 400 mg BID and trametinib 0.5 mg QD or Compound A 200 mg BID and trametinib 1 mg QD
   Arm 3: Compound A 400 mg BID and ribociclib 400 mg QD or Compound A 200 mg BID and ribociclib 400 mg QD
Group 2: Participants with NRAS mutant unresectable or metastatic melanoma:
   Arm 4: Compound A 400 mg BID and Compound B 200 mg QD or Compound A 200 mg BID (twice a day) and Compound B 200 mg QD (once a day)
   Arm 5: Compound A 400 mg BID and trametinib 0.5 mg QD or Compound A 200 mg BID and trametinib 1 mg QD
   Arm 6: Compound A 400 mg BID and ribociclib 400 mg QD or Compound A 200 mg BID and ribociclib 400 mg QD

Each cycle is 28 days and all drugs are administered orally and given continuously with the exception of ribociclib, which is given 3 weeks on/1 week off.

### Part 2: Expansion

Part 2 further characterizes the efficacy of LXH254 combinations that were deemed safe and potentially efficacious as determined in Part 1. Additional participants may be enrolled to further evaluate the efficacy of the combinations tested in Part 1. Dosages and dosing regimens as described in Part 1 may be used in Part 2.

### Study Treatment

Each cycle is 2.8 days. All study drugs may be administered orally and continuously except ribociclib, which may be administered daily for 21 days followed by one week off.
Examples of dosing regimens are as follows:
Compound A 400 mg BID in combination with Compound B 200 mg OD;
Compound A 200 rag BID in combination with Compound B 200 mg QD;
Compound A 400 mg BID in combination with trametinib 0.5 mg QD;
Compound A 200 mg BID in combination with trametinib 1 mg QD;
Compound A 400 mg BID in combination with ribociclib 400 mg QD:
Compound A 200 mg BID in combination with ribociclib 400 mg QD;

Typical dosages and regimens are as follows

| Therapeutic | Pharmaceutical form and | | Frequency and/or |
|---|---|---|---|
| agent | route of administration | Dose | Regimen |
| Compound A | Tablet for oral use | 400 mg | Twice daily (BID), |
| | | | continuous |
| Compound B | Capsule for oral use | 200 mg | Once daily (QD), |
| | | | continuous |
| trametinib | Tablet for oral use | 0.5 mg | QD, continuous |
| (Compound C) | | | |
| Ribociclib | Tablet for oral use | 400 mg | QD, preferably 3 |
| (Compound D) | | | weeks on/1 week |
| | | | off |

Other typical dosages and regimens include:

| Therapeutic | Pharmaceutical form and | | Frequency and/or |
|---|---|---|---|
| agent | route of administration | Dose | Regimen |
| Compound A | Tablet for oral use | 200 mg | Twice daily (BID), |
| | | | continuous |
| Compound B | Capsule for oral use | 200 mg | Once daily (QD), |
| | | | continuous |
| trametinib | Tablet for oral use | 1 mg | QD, continuous |
| (Compound C) | | | |
| Ribociclib | Tablet for oral use | 400 mg | QD, preferably 3 |
| (Compound D) | | | weeks on/1 week |
| | | | off |

Dose reduction may be possible, e.g., due to toxicity, while maintaining clinical efficacy

| | | |
|---|---|---|
| Starting dose | | |
| level - 0 | Dose level - 1 | Dose level - 2 |

Dose reduction steps for Compound A plus Compound B*

| | | | |
|---|---|---|---|
| Compound A (BID) | 400 mg | 200 mg | 100 mg |

Dose reduction*

| | | | |
|---|---|---|---|
| Compound B (Daily) | 200 mg | 150 mg | 100 mg |

*Dose reduction based on the worst toxicity demonstrated at the last dose.

| | | |
|---|---|---|
| Starting dose | | |
| level - 0 | Dose level - 1 | Dose level - 2 |

Dose reduction steps for Compound A plus Compound C*

| | | | |
|---|---|---|---|
| Compound A (BID) | 400 mg | 200 mg | 100 mg |

Dose reduction*

| | | | |
|---|---|---|---|
| trametinib (Daily) | 0.5 mg | 0.5 mg | 0.5 mg |

*Dose reduction based on the worst toxicity demonstrated at the last dose.

| | | |
|---|---|---|
| Starting dose | | |
| level - 0 | Dose level - 1 | Dose level - 2 |

Dose reduction steps for Compound A plus Compound D*

| | | | |
|---|---|---|---|
| Compound A (BID) | 400 mg | 200 mg | 100 mg |

Dose reduction*

| | | | |
|---|---|---|---|
| Ribociclib (Daily) | 400 mg | 200 mg | 100 mg |

*Dose reduction based on the worst toxicity demonstrated at the last dose.

Especially hand foot skin reaction, nausea and vomiting, decrease of left ventricular ejection fraction, rash are signs of toxicity.
Adverse events are assessed and graded according to the Common Terminology Criteria for Adverse Events (CTCAE) version 5.0.
To ensure participant safety, every Severe Adverse Event (SAE), regardless of causality, occurring after the participant has provided informed consent and until 30 days must be reported to Novartis safety within 24 hours of learning of its occurrence.

### Efficacy Assessments

Radiological tumor assessments by local RECIST v1.1:
   At screening
   During treatment: every 8 weeks (±7 days) after the date of randomization thereafter until disease progression per RECIST v1.1 (as assessed by the investigator), death, lost to follow-up or withdrawal of consent
   End of Treatment (EOT): Only to be done if a scan was not conducted within 30 days prior to end of study treatment
   Efficacy follow-up: If a participant discontinues study treatment for a reason other than disease progression per RECIST v1.1, efficacy follow-up must be continued on the same schedule as during treatment until disease progression per RECIST v1.1, even if additional antineoplastic therapy has been initiated.

ORR may be determined according to methods known in the art, e.g., using any one or more of the methods used in the following table:

| Procedure | Screening/Baseline | During Treatment/Follow-up |
|---|---|---|
| Chest, abdomen and pelvis | Mandated | Mandated, every 8 weeks (+/−7 |
| CT or MRI (with | | days) |
| intravenous contrast | | |
| enhancement) | | |
| Brain CT or MRI | If clinically indicated | If lesions were documented at |
| | | baseline, follow same schedule |
| | | as CT/MRI of chest, abdomen, |
| | | and pelvis |
| Whole body bone scan | If clinically indicated | If clinically indicated |
| Localized bone CT, MRI or | For any lesions identified on the | If lesions were documented at |
| x-ray | whole body bone scan that are | baseline, follow same schedule |
| | not visible on the chest, abdomen | as CT/MRI of chest, abdomen, |
| | and pelvis CT or MRI | and pelvis |
| Color photography (with | For any skin lesions present | If lesions were documented at |
| scale/ruler) | | baseline, follow same schedule |
| | | as CT/MRI of chest, abdomen, |
| | | and pelvis |
| CT or MRI of other | If clinically indicated | If lesions were documented at |
| metastatic sites (e.g., neck) | | baseline, follow same schedule |
| | | as CT/MRI of chest, abdomen, |
| | | and pelvis |

All complete responses (CR) and partial responses (PR) must be confirmed by a second assessment, If this second assessment is performed outside the normal window of scans, it should not be performed earlier than 4 weeks after the scan where the criteria for response are first met.
Best overall response is the best post baseline confirmed overall response observed in a given participant, among the confirmed response categories, excluding “unknown” and “not assessed”. BOR is defined as the best response recorded from the start of the randomization until disease progression/recurrence death, start of new therapy, withdrawal of consent or end of study, whatever comes first as per central assessment and according to RECIST v1.1.

The primary objective of this study is to evaluate the efficacy of each combination arm, as measured by confirmed objective response rate (ORR) by local investigator's assessment per RECIST v1.1 (as defined also above).

### Secondary Objectives Are

to characterize the safety and tolerability of each combination arm through the incidence and severity of adverse (AEs) including changes in laboratory values, vital signs, cardiac assessment, dose interruptions, reduction and permanent discontinuations of study treatments;
to further evaluate the efficacy of each combination arm by duration of response (DOR), progression free survival (PFS) and disease control rate (DCR) using RECIST v1.1, per local assessment. Additionally, for the treatment combination arm(s) that expands, DOR, PFS, DCR and ORR are evaluated using RECIST v1.1 per central assessment;
to evaluate the overall survival (OS) of each combination arm and
to characterize the pharmacokinetics of each combination regimen through serum/plasma concentration and pharmacokinetic parameters of each combination regimen.

### Population

Adults (≥18 years) and adolescent (12-17 years) participants with BRAFV600 or NRAS mutant unresectable or metastatic melanoma previously treated with a therapy which is selected from treatment with talimogene laherparepvec (T-vec), immunotherapy (CPI), and targeted therapy (e.g. RAF+/−MEK inhibitors), and combinations thereof. The previous therapy may be for example treatment with checkpoint inhibitors (CPI) and RAF+/−MEK inhibitors or CPI only.

### Key Inclusion Criteria Comprise

a Male or female must be 12 years
For adolescent participants only (12-17 years): body weight >40 kg
Histologically confirmed unresectable or metastatic cutaneous melanoma
Documentation of BRAFV600 or NRAS mutation in tumor tissue prior to study treatment as determined by local assay agreed by Novartis or as determined by central pre-screening assessment performed at a Novartis designated laboratory.
   Previously treated for unresectable or metastatic melanoma:

### Participants with NRAS Mutation Include

Participants must have received prior systemic therapy for unresectable or metastatic melanoma with an anti-PD-1/PD-L1 checkpoint inhibitor as a single agent or in combination with anti-CTLA-4. No additional systemic treatment is allowed for unresectable or metastatic melanoma
A maximum of two prior lines of systemic immunotherapy for unresectable or metastatic melanoma are allowed
The last dose of prior therapy (anti-PD-1, anti-PD-L1 or anti-CTLA-4 must have been received more than four weeks before randomization
Participants must have documented confirmed progressive disease as per RECIST v1.1 while on/after treatment with checkpoint inhibitor therapy. The last progression must have occurred within 12 weeks prior to randomization in the study

### Participants With BRAFV600 Mutant Disease Include

Participants must have received prior systemic therapy for unresectable or metastatic melanoma with anti-PD-1 or an PD-L1 as a single agent or in combination with anti-CTLA-4. Additionally, participants must have received targeted therapy with a RAFi as a single agent or in combination with a MEKi as the last prior therapy. No additional systemic treatment is allowed for advanced or metastatic melanoma
A maximum of three prior lines of systemic therapy for unresectable or metastatic melanoma are allowed
The last dose of targeted therapy (last prior therapy) must have been received more than 2 weeks prior to randomization

Participants must have documented progressive disease as per RECIST v1.1 while on/after treatment with targeted therapy. The last progression must have occurred within 12 weeks prior to randomization in the study
Participants must have a site of disease amenable to repeated biopsies and must be willing to undergo a new tumor biopsy at baseline and during treatment according to the treating institution's own guidelines and requirements for such procedure. Bone metastases are not acceptable as a site for biopsy.

### Key Exclusion Criteria Comprise

All primary central nervous system(CNS) tumors or symptomatic CNS metastases that are neurologically unstable
Insufficient bone marrow, hepatic or renal function at the screening visit
Abnormal electrocardiogram (ECU) as determined by the mean of a triplicate ECU and assessed locally
Cardiac disease or cardiac repolarization abnormality
Presence of ≥CTCAE grade 2 toxicity (except alopecia and ototoxicity, which are excluded if ≥CTCAE grade 3) due to prior anti-melanoma therapy
History or current evidence of retinal vein occlusion (RVO) or current risk factors for RVO (e.g. uncontrolled glaucoma or ocular hypertension, history of hyperviscosity or hypercoagulability syndromes)

The inclusion and or exclusion criteria may also be modified. For example, patients in all aims and groups who have other prior therapies (e.g. received talimogene laherparepvec (T-vec) as well as investigational agents administered with CPI) may also be included in the clinical trial. In addition, prior therapy with immunotherapy (CPI) in the metastatic setting may not be mandated in patients who have progressed on or within 6 months of adjuvant CPI therapy.

Preliminary data from a clinical trial carried out according to the methods and uses described herein are detailed below.

Out of 18 patients treated with a combination of Compound A and ribociclib, the response from four patients who have so far received and tolerated 6 cycles of combination treatment were classified as “Stable Disease (SD)”. The response from a further 8 patients was classified as unconfirmed “Stable Disease” status and 6 patients suffered progressive disease (PD).

The table below shows that a combination of Compound A and trametinib may be especially beneficial for treating NRAS-mutant melanoma, especially when Compound A is administered at a dose of 200 mg BID and trametinib is administered at a dose of 1 mg QD. This combination and this dosing administration gives rise to an improved efficacy (20 fold increase in ORR when compared to a dose of 400 mg BID of naporafenib and a dose of 0.5 mg QD of trametinib) and is likely to be accompanied by a lower risk of adverse side-effects.

The Table below shows the efficacy of a combination of naporafenib and trametinib in NRAS-mutant melanoma.

TABLE 5
Best overall response per RECIST 1.1 based on Investigator's
assessment with confirmation by cobort, Compound A (LXH254) +
trametinib (TMT) NRAS melanoma patients Full analysis set

| | LXH254 | LXH254 | |
|---|---|---|---|
| | 200 mg BID + | 400 mg BID + | |
| | trametinib | trametinib | |
| | 1 mg QD | 0.5 mg QD | All Patients |
| | N = 15 | N = 17 | N = 32 |
| Best overall response | n (%) | n (%) | n (%) |
| Complete Response (CR) | 0 | 0 | 0 |
| Partial Response (PR) | 6 (40.0) | 2 (11.8) | 8 (25.0) |
| Stable Disease (SD) | 5 (33.3) | 8 (47.1) | 13 (40.6) |
| Progressive Disease (PD) | 4 (26.7) | 4 (23.5) | 8 (25.0) |
| Unknown (UNK) | 0 | 3 (17.6) | 3 (9.4) |
| Overall Response Rate | 6 (40.0) | 2 (11.8) | 8 (25.0) |
| (ORR: CR + PR) | | | |
| 95% CI ORR | (16.3, 67.7) | (1.5, 36.4) | (11.5, 43.4) |
| Disease Control Rate | 11 (73.3) | 10 (58.8) | 21 (65.6) |
| (DCR: CR + PR + SD) | | | |
| 95% CI DCR | (44.9, 92.2) | (32.9, 81.6) | (46.8, 81.4) |

N: The total number of subjects in the treatment group. It is the denominator for percentage (%) calculation.
n: Number of patients who are at the corresponding category.
The 95% CI for the frequency distribution of each variable were computed using Clopper-Pearson.

1.-4. (canceled)

5. A method of treating NRAS-mutant melanoma and/or BRAF-mutant melanoma in a patient having said disease comprising administering to the patient in need thereof a therapeutically effective amount of a pharmaceutical combination comprising the Compound of formula (I) (naporafenib), 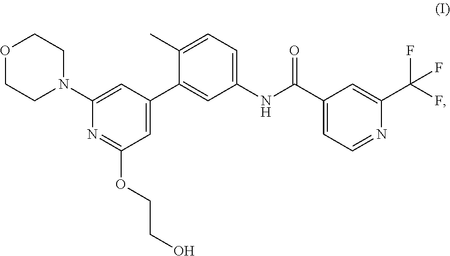 [SMILES: Cc1ccc(NC(=O)c2ccnc(C(F)(F)F)c2)cc1-c1cc(OCCO)nc(N2CCOCC2)c1]
or a pharmaceutically acceptable salt thereof,
and a second therapeutic agent which is trametinib, or a pharmaceutically acceptable salt or solvate thereof wherein the melanoma is unresectable and/or metastatic cutaneous melanoma.

6. The method according to claim 5, wherein the melanoma is NRAS-mutant melanoma.

7. The method according to claim 6, wherein the NRAS-mutant melanoma is a mutation selected from the group consisting of G12C, G12R, G12D, G12V, G12S, G12A, G13R, G13D, G13C, G13A, G13, G13S, G13V, Q61R, Q61L, Q61K, Q61H, Q61P, Q61E and combinations thereof.

8 The method according to claim 5, wherein the melanoma is BRAT-mutant melanoma.

9. The method according to claim 8 wherein the melanoma is BRAFD287H-mutant melanoma or BRAFV600-mutant melanoma.

10. The method according to claim 8, wherein the melanoma is BRAFV600E-mutant, BRAFV600K-mutant melanoma, BRAFV600R-mutant or BRAFV600D-mutant.

11. The method according to claim 5, wherein the melanoma is refractory or resistant to chemotherapy, e.g., dacarbazine.

12. The method according to claim 5, wherein the patient suffering from the melanoma to be treated has received prior therapy, optionally wherein the prior therapy is selected from the group consisting of:
treatment with talimogene laherparepvec;
standard care chemotherapy;
treatment with a cytotoxic agent such as a nitrosurea and/or mitomycin C;
immunotherapy;
treatment with an anti-PD-1 or an PD-L1 checkpoint inhibitor as a single agent or in combination with anti-CTLA-4;
targeted therapy;
and combinations thereof.

13. The method according to claim 5, wherein the melanoma to be treated is resistant or refractory to treatment with immunotherapy.

14. The method according to claim 5, wherein the patient suffering from the melanoma to be treated has received prior therapy with a RAF-inhibitor as a single agent or in combination with a MEK-inhibitor.

15. The method according to claim 14 wherein the RAF inhibitor is selected from one of more from the group consisting of dabrafenib, vemurafenib and encorafenib.

16. The method according to claim 14, wherein the MEK inhibitor is selected from one of more from the group consisting of trametinib, cobimetinib and binimetinib.

17. The method according to claim 14, wherein the patient suffering from the melanoma to be treated has received prior therapy with a combination which is selected from (i) dabrafenib and trametinib, (ii) vemurafenib and cobimetinib, and (iii) encorafenib and binimetinib.

18. The method according to claim 5, wherein the total daily dose of naporafenib or trametinib is administered either QD (once daily) or BID (twice daily).

19. The method according to claim 5, wherein naporafenib or trametinib is administered continuously.

20. The method according to claim 5, wherein trametinib is administered intermittently.

21. (canceled)

22. The method according to claim 5 wherein the total daily dose of naporafenib is from about 200 mg to about 800 mg.

23. The method according to claim 5, wherein
the total daily dose of trametinib is from about 0.5 mg to about 1 mg.

24. (canceled)

25. (canceled)

26. Naporafenib, or a pharmaceutically acceptable salt thereof, for use in treating NRAS-mutant or BRAF-mutant melanoma, wherein naporafenib is administered in a total daily dose of about 800 mg and trametinib, or a pharmaceutically acceptable solvate thereof, is further administered in a total daily dose of about 0.5 mg or about 1.0 mg, wherein the melanoma is unresectable and/or metastatic melanoma.

27. Naporafenib, or a pharmaceutically acceptable salt thereof, for use in treating NRAS-mutant or BRAF-mutant melanoma, wherein naporafenib is administered in a total daily dose of about 400 mg and trametinib, or a pharmaceutically acceptable solvate thereof, is further administered in a total daily dose of about 0.5 mg or about 1.0 mg, optionally wherein the melanoma is unresectable and/or metastatic melanoma.

28.-31. (canceled)

32. The method for use according to claim 5 wherein naporafenib is administered at a dose of 200 mg twice a day and trametinib is administered at a dose of 1 mg per day.

33.-36. (canceled)
